(19) United States
(12) Patent Application Publication
(10) Pub. No.: US 2025/0312381 A1
(43) Pub. Date: Oct. 9, 2025

(54) METHODS OF INTESTINAL INJURY REPAIR USING ORGANOID COMPOSITIONS

(71) Applicant: Children's Hospital Medical Center, Cincinnati, OH (US)

(72) Inventors: Holly M. Poling, Cincinnati, OH (US); Michael A. Helmrath, Cincinnati, OH (US); Nambirajan Sundaram, Mason, OH (US); James M. Wells, Cincinnati, OH (US); Maxime Mickael Mahe, Reze (FR)

(73) Assignee: Children's Hospital Medical Center, Cincinnati, OH (US)

(21) Appl. No.: 18/728,383

(22) PCT Filed: Jan. 13, 2023
(86) PCT No.: PCT/US23/60687

§ 371 Date: Jul. 11, 2024

Related U.S. Application Data
(60) Provisional application No. 63/299,842, filed on Jan. 14, 2022.

(51) Int. Cl. A61K 35/38; A61P 1/00; C12N 5/071

(52) U.S. Cl. CPC A61K 35/38; A61P 1/00; C12N 5/0679

(57) ABSTRACT

Disclosed herein are organoid compositions and methods of use thereof for the treatment of intestinal injury and damage. The methods involve the intraluminal administration of cellular compositions derived from stem cell-derived organoids, which comprise both epithelial and mesenchymal components. The administered cells show robust engraftment into the appropriate regions of the recipient intestinal tissue. Integration of the multiple cell types present from the stem cell-derived organoids results in a more complete healing of the intestinal injury.

### RELATED APPLICATIONS

This application is a U.S. National Stage application under 35 U.S.C. 371 of International Application No. PCT/US2023/060687, filed Jan. 13, 2023, which claims priority to and the benefit of U.S. Provisional Application No. 63/299,842, filed Jan. 14, 2022, the contents of which are incorporated into the present application by reference in their entireties.

### STATEMENT REGARDING FEDERALLY SPONSORED R&D

This invention was made with government support under U01 DK103117 and NIH P30 DK078292, awarded by the National Institutes of Health. The government has certain rights to the invention.

### REFERENCE TO SEQUENCE LISTING

The present application is being filed along with a Sequence Listing in electronic format. The Sequence Listing is provided as a file entitled CHMC63_045WO.xml which was created and last modified on Jan. 12, 2023, which is 6021 bytes in size. The formation in the electronic Sequence Listing is hereby incorporated by reference in its entirety.

### FIELD OF THE INVENTION

Aspects of the present disclosure relate generally to organoid compositions and methods of use thereof for the treatment of intestinal damage.

### BACKGROUND

A wide range of enteropathies are associated with ulceration of the intestinal tissue. Common indications resulting in and/or associated with intestinal ulcers include Crohn's disease, ulcerative colitis, enteropathies associated with non-steroidal anti-inflammatory drugs (NSAIDs) and other medications, radiation-induced enteropathies, and those associated with pathogenic infections including tuberculosis. However, chronic ulceration of the intestinal tract with obscure causes have also been reported. While detection of intestinal ulcers have improved with the use of endoscopic approaches such as capsule endoscopy and balloon endoscopy, there still lacks a straightforward approach for the treatment of these ulcers. While in some situations, improvement may be seen after termination of use of a suspected medication with associated side effects, surgical resection may be necessary in advanced cases. Chronic intestinal damage resulting in non-healing ulcerated regions of the intestine pose a clinical challenge as fully restorative treatment options are currently lacking. Accordingly, there is a lasting need for therapies that can be broadly applicable to different manifestations of intestinal ulceration and injury with long-lasting effect.

Peptic Ulcer Disease (PUD), commonly referred to as stomach or duodenal ulcers, is a chronic, potentially life-threatening condition characterized by erosion of the small intestinal epithelium. Commonly caused by bacteria or the overuse of nonsteroidal anti-inflammatory drugs (NSAIDs), PUD affects up to 10% of the general population and has a fatality rate of up to 10%. Although recent advances in H. pylori treatment and more careful use of NSAIDs has decreased the incidence of PUD, overall mortality rates have failed to decrease. In fact, the incidence of idiopathic bleeding ulcers, associated with high mortality, has increased. Treatment plans for PUD have become increasingly complicated and represent an opportunity for novel therapy advancement. H. pylori antibiotic resistance has been on the rise, and conventional pharmacological treatments can result in numerous adverse side effects without fully restoring the ulcerated region to a healthy tissue state. To broaden the treatment modalities available for these patients, as well as to develop treatments for those with idiopathic bleeding ulcers, it is important to develop novel therapeutic strategies. Development of these strategies could also be extended to the management and treatment of other major conditions, including Crohn's disease, ulcerative colitis, and chronic ulceration disorders.

### SUMMARY

The disclosure herein relates to dissociated cell populations or compositions derived from intestinal organoids, colonic organoids, or both, as well as methods of using them to treat intestinal damage in a subject. These intestinal organoids and colonic organoids are derived from pluripotent stem cells, such as embryonic stem cells or induced pluripotent stem cells, such that the organoids are made up of many cell types normally found in the small intestine and colon, particularly containing both epithelial and mesenchymal cell types. The dissociated cell populations or compositions are produced through the dissociation or fragmentation of the intestinal organoid and colonic organoids, and in some embodiments, the dissociated cell populations or compositions are made up entirely or mostly of clumps of live cells (also referred to herein as “fragments”), which represent fragments of the intestinal organoid or colonic organoids. The dissociated cell populations or compositions are administered to the luminal wall of the intestine of the subject, which can be done through a variety of approaches. The presence of mesenchymal cells in the dissociated cell populations or compositions results in superior engraftment into the intestine of the subject, and healing of the intestinal damage in the subject.

Disclosed herein are methods of treating intestinal damage of a subject in need thereof. In some embodiments, the methods comprise administering a dissociated cell population that is dissociated from intestinal and/or colonic organoids to the luminal wall of the intestine of the subject. In some embodiments, the dissociated cell population comprise epithelial cell types and mesenchymal cell types. In some embodiments, the intestine of the subject comprises the small intestine and/or the colon.

Also disclosed herein are methods of treating intestinal damage of a subject in need thereof. In some embodiments, the methods comprise producing intestinal and/or colonic organoids comprising epithelial cell types and mesenchymal cell types, dissociating the intestinal and/or colonic organoids to produce a cell population comprising the epithelial cell types and mesenchymal cell types, and administering the cell population to the lumen of the intestine of the subject. In some embodiments, the intestine of the subject comprises the small intestine and/or the colon.

Also disclosed herein are dissociated cell populations that are dissociated from intestinal and/or colonic organoids for use in a method of treating a gastrointestinal malady in a subject in need thereof. In some embodiments, the method comprises administering the dissociated cell population to the luminal wall of the intestine of the subject. In some embodiments, the dissociated cell population comprises epithelial cell types and mesenchymal cell types. In some embodiments, the intestine of the subject comprises the small intestine and/or the colon.

Also disclosed herein are cell suspensions comprising dissociated cell populations comprising epithelial cell types and mesenchymal cell types. Also disclosed herein are pharmaceutical formulations comprising any of the cell suspensions or dissociated cell populations disclosed herein.

Embodiments of the present disclosure provided herein are described by way of the following numbered embodiments:

1. A method of treating intestinal damage of a subject in need thereof, comprising administering a dissociated cell population that is dissociated from intestinal and/or colonic organoids to the luminal wall of the intestine of the subject, wherein the cell population dissociated from the intestinal and/or colonic organoids comprise epithelial cell types and mesenchymal cell types, and wherein the intestine of the subject comprises the small intestine and/or the colon.

2. The method of embodiment 1, wherein administering the dissociated cell population to the luminal wall of the intestine of the subject comprises administering the cell population to a location of the lumen of the intestine affected by the intestinal damage, optionally wherein the location is directly adjacent to or near the intestine affected by the intestinal damage, optionally to the surface of the luminal wall.

3. The method of embodiment 1 or 2, wherein the dissociated cell population is administered to the luminal wall of the intestine of the subject as a cell suspension.

4. The method of any one of embodiments 1-3, wherein administering the dissociated cell population to the luminal wall of the intestine of the subject comprises administering the cell population by oroenteric catheter, nasoenteric catheter, or enema.

5. The method of any one of embodiments 1-4, wherein the intestinal and/or colonic organoids have been derived from precursor cells selected from embryonic stem cells, induced pluripotent stem cells, and definitive endoderm cells.

6. The method of any one of embodiments 1-5, wherein the intestinal and/or colonic organoids are allogeneic to the subject.

7. The method of any one of embodiments 1-5, wherein the intestinal and/or colonic organoids have been derived from cells isolated from the subject, and the intestinal and/or colonic organoids are autologous to the subject.

8. The method of embodiment 7, wherein the intestinal and/or colonic organoids have been derived from induced pluripotent stem cells derived from the cells isolated from the subject.

9. The method of embodiment 8, wherein the cells isolated from the subject comprise dermal fibroblasts or peripheral blood mononuclear cells (PBMCs) from the subject.

10. The method of any one of embodiments 1-9, wherein the dissociated cell population are prepared by enzymatic dissociation and/or mechanical dissociation of the intestinal and/or colonic organoids.

11. The method of embodiment 10, wherein enzymatic dissociation comprises dissociating the intestinal and/or colonic organoids with trypsin, chymotrypsin, collagenase, papain, hyaluronidase, elastase, thermolysin, neutral protease, or any combination thereof.

12. The method of embodiment 10 or 11, wherein mechanical dissociation comprises passing the intestinal and/or colonic organoids through successively narrower bore channels.

13. The method of any one of embodiments 1-12, wherein the percentage of cells in the dissociated cell population that are mesenchymal cell types is or is about 85%, 86%, 87%, 88%, 89%, 90%, 91%, 92%, 93%, 94%, or 95%, or any percentage within a range defined by any two of the aforementioned percentages.

14. The method of any one of embodiments 1-13, wherein the percentage of cells in the dissociated cell population that are epithelial cell types is or is about 5%, 6%, 7%, 8%, 9%, 10%, 11%, 12%, 13%, 14%, or 15%, or any percentage within a range defined by any two of the aforementioned percentages.

15. The method of any one of embodiments 1-14, wherein the dissociated cell population is administered at a concentration that is or is about 50, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 cells per mm² of affected intestine surface area, or any amount of cells per mm² within a range defined by any two of the aforementioned values.

16. The method of any one of embodiments 1-15, wherein the dissociated cell population is administered to the subject for a number of times until an improvement in the intestinal damage is observed.

17. The method of embodiment 16, wherein the dissociated cell population is administered to the subject 1, 2, 3, 4, 5, 6, 7, 8, 9, or 10 times.

18. The method of any one of embodiments 1-17, wherein cells of the dissociated cell population integrate into the mucosa and muscularis of the intestine of the subject.

19. The method of embodiment 18, wherein cells of the dissociated cell population integrated into the intestine of the subject maintain their intestinal and/or colonic regionality.

20. The method of embodiment 18 or 19, wherein cells, or a subpopulation thereof, of the dissociated cell population integrated into the intestine of the subject differentiate into smooth muscle actin (SMA)-positive smooth muscle cell types.

21. The method of any one of embodiments 1-20, wherein the dissociated cell population comprises Marker of Proliferation KI67+ (MKI67+) proliferative cells that integrate into the intestine of the subject and promote healing of the intestinal damage.

22. The method of any one of embodiments 1-21, wherein the dissociated cell population promotes formation of an intact intestinal barrier after administration.

23. The method of any one of embodiments 1-22, wherein the dissociated cell population integrates into at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% of the surface area of the luminal wall of the intestine of the subject affected by the intestinal damage, or any percentage of surface area within a range defined by any two of the aforementioned percentages.

24. The method of any one of embodiments 1-23, wherein the intestinal damage comprises intestinal ulceration.

25. The method of any one of embodiments 1-24, wherein the intestinal damage is chemical and/or mechanical.

26. The method of any one of embodiments 1-25, wherein the intestinal damage is associated with a gastrointestinal malady.

27. The method of embodiment 26, wherein the gastrointestinal malady is selected from Crohn's disease, ulcerative colitis, enteropathies associated with non-steroidal anti-inflammatory drugs (NSAIDs) or other medications, radiation-induced enteropathies, and enteropathies associated with pathogenic infections such as tuberculosis.

28. The method of any one of embodiments 1-27, wherein the intestinal and/or colonic organoids are mammalian.

29. The method of any one of embodiments 1-28, wherein the intestinal and/or colonic organoids are human.

30. The method of any one of embodiments 1-29, wherein the subject is mammalian.

31. The method of any one of embodiments 1-30, wherein the subject is human.

32. The method of any one of the preceding embodiments, further comprising dissociating the intestinal and/or colonic organoids to produce the dissociated cell population.

33. The method of any one of the preceding embodiments, wherein the dissociated cell population is in the form of 30%, 35%, 40%, 45%, 50%, 55%, 60%, 65%, 70%, 75%, 80%, 85%, 90%, 95%, 96%, 97%, 98%, 99%, or 100% multi-cellular fragments, or in the form of at least 30%, 35%, 40%, 45%, 50%, 55%, 60%, 65%, 70%, 75%, 80%, 85%, 90%, 95%, 96%, 97%, 98%, 99%, or 100% multi-cellular fragments.

34. The method of any one of the preceding embodiments, further comprising producing the intestinal and/or colonic organoids in vitro, optionally from pluripotent stem cells.

35. A dissociated cell population dissociated from intestinal and/or colonic organoids for use in the method of any one of the preceding embodiments.

36. A cell suspension comprising a dissociated cell population comprising epithelial cell types and mesenchymal cell types.

37. The cell suspension of embodiment 36, wherein the mesenchymal cell types express vimentin (VIM) and/or elastin microfibril interfacer 1 (EMILIN1); and the epithelial cell types express E-cadherin (CDH1) and/or caudal type homeobox 2 (CDX2).

38. The cell suspension of embodiment 36 or 37, wherein the dissociated cell population is dissociated from intestinal and/or colonic organoids, wherein the intestinal and/or colonic organoids comprise epithelial cell types and mesenchymal cell types.

39. The cell suspension of embodiment 38, wherein the intestinal and/or colonic organoids have been derived from precursor cells selected from embryonic stem cells, induced pluripotent stem cells, and definitive endoderm cells.

40. The cell suspension of any one of embodiments 35-39, wherein the cell suspension or the intestinal and/or colonic organoids are allogeneic to a subject.

41. The cell suspension of any one of embodiments 35-40, wherein the cell suspension or the intestinal and/or colonic organoids have been derived from cells from a subject, and the intestinal and/or colonic organoids are autologous to the subject.

42. The cell suspension of embodiment 41, wherein the cell suspension or the intestinal and/or colonic organoids have been derived from induced pluripotent stem cells derived from the cells isolated from the subject.

43. The cell suspension of any one of embodiments 35-42, wherein the dissociated cell population are prepared by enzymatic dissociation and/or mechanical dissociation of the intestinal and/or colonic organoids.

44. The cell suspension of embodiment 43, wherein enzymatic dissociation comprises dissociating the intestinal and/or colonic organoids with trypsin, chymotrypsin, collagenase, papain, hyaluronidase, elastase, thermolysin, neutral protease, or any combination thereof.

45. The cell suspension of embodiment 43 or 44, wherein mechanical dissociation comprises passing the intestinal and/or colonic organoids through successively narrower bore channels.

46. The cell suspension of any one of embodiments 35-45, wherein the dissociated cell population comprises MKI67+ proliferative cells.

47. The cell suspension of any one of embodiments 35-46, wherein the percentage of cells in the dissociated cell population that are mesenchymal cell types is at least 85%, 86%, 87%, 88%, 89%, 90%, 91%, 92%, 93%, 94%, or 95%, or a percentage within a range defined by any two of the aforementioned percentages.

48. The cell suspension of any one of embodiments 35-47, wherein the percentage of cells in the dissociated cell population that are epithelial cell types is no more than 5%, 6%, 7%, 8%, 9%, 10%, 11%, 12%, 13%, 14%, or 15%, or a percentage within a range defined by any two of the aforementioned percentages.

49. The cell suspension of any one of embodiments 35-48, wherein the concentration of the dissociated cell population in the cell suspension is about 10⁵, 10⁶, 10⁷, 10⁸, 10⁹, 10¹⁰, or 10¹¹ cells/mL, or any concentration of cells within a range defined by any two of the aforementioned concentrations.

50. The cell suspension of any one of embodiments 35-49, wherein the concentration of cells in the dissociated cell population that are mesenchymal cell types is about 10⁵, 10⁶, 10⁷, 10⁸, 10⁹, 10¹⁰, or 10¹¹ cells/mL, or any concentration of cells within a range defined by any two of the aforementioned concentrations.

51. The cell suspension of any one of embodiments 35-49, wherein the dissociated cell population is in the form of, or of at least, 30%, 35%, 40%, 45%, 50%, 55%, 60%, 65%, 70%, 75%, 80%, 85%, 90%, 95%, 96%, 97%, 98%, 99%, or 100% multi-cellular fragments, or any percentage within a range defined by any two of the aforementioned percentages.

52. A pharmaceutical composition comprising an effective amount of the cell suspension of any one of embodiments 35-51, and at least one pharmaceutically acceptable carrier, excipient, or diluent.

53. The cell suspension of any one of embodiments 35-51 or the pharmaceutical composition of embodiment 52 for use in the treatment of intestinal damage.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1A-H depict an embodiment of dissociated HIOs that contribute to tissue regeneration of damaged bowel in vivo. FIG. 1A depicts an embodiment of a schematic of the experimental design. FIG. 1B depicts brightfield (left panel) and GFP (right panel) images of a harvested reseeded loop. Live-GFP presence demonstrates GFP-HIO fragments incorporated within the loop ten weeks post-operation. Black and white dashed lines indicate the perimeter of the loop. Dashed lines within the white dashed lines of the GFP image indicates the perimeters of live-GFP expressing regions within the loop. Scale bar=1 mm. FIG. 1C depicts an embodiment of a quantification of live-GFP expressing regions within loops, as pictured in FIG. 1B. Violin plot of loop surface area expressing live-GFP as a percentage of the whole loop; n=7. FIG. 1D depicts an embodiment of immunostaining for human cells within a dissected region with live-GFP presence, as pictured in FIG. 1B. FIG. 1E depicts an embodiment of brightfield (left) and GFP (right) images of a harvested loop reseeded with fragmented enteroids. Live-GFP presence demonstrates GFP-Enteroid fragments incorporated within the loop ten weeks post-operatively. Black and white dashed lines indicate the perimeter of the loop. Scale bar=1 mm. FIG. 1F depicts an embodiment of quantification of live-GFP expressing regions within loops, as pictured in FIG. 1E. Violin plot of loop surface area expressing live-GFP as a percentage of the whole loop; n=7. FIG. 1G depicts an embodiment of immunostaining for human cells within a dissected region with live-GFP presence from FIG. 1E. FIG. 1H depicts an embodiment of a representative tile scan of a damaged sham loop stained for a human specific marker (KU80) and nuclei (hematoxylin); scale bar=0.5 cm; n=3. No cells of human origin were observed throughout the tissue of the sham loop.

FIG. 2A depicts an embodiment of a representative tile scan of a damaged and reseeded loop stained for a human specific marker (KU80) and nuclei (hematoxylin); scale bar=0.5 cm; n=7. Cells of human origin were observed throughout the reseeded loop. FIG. 2B depicts an embodiment of higher magnification images of the mucosal and muscularis regions in FIG. 2A; scale bar=100 μm. FIG. 2C depicts an embodiment of representative images of sham operated loops and reseeded loops stained for Actin Alpha 2, Smooth Muscle (ACTA2), GFP, and nuclei (DAPI); scale bar=50 μm; n=3 for both groups. FIG. 2D depicts an embodiment of representative images of sham operated loops and reseeded loops stained for Tubulin Beta 3 Class III (TUBB3) and nuclei (hematoxylin); scale bar=50 μm; n=3 for both groups. Arrowheads indicate neuronal bundles from the rat host.

FIG. 3A depicts an embodiment of an HI-GFP cell line retaining a normal karyotype after gene editing. G-banded karyotype analysis demonstrates normal (46, XY) karyotype of H1 embryonic stem cell line after GFP insertion. FIG. 3B depicts an embodiment of an electropherogram of short tandem repeat analysis of HI embryonic stem cell line after GFP insertion displaying pass result.

FIG. 4 depicts an embodiment of photographs of key steps during the mucosectomy surgery. Panel A depicts loop creation and anastomosis; arrowhead marks the anastomosis site. Panel B depicts chemically damaging the loop from the open proximal end, while the distal end is closed with a bulldog clamp. Panel C depicts mechanically damaging the loop with a dental go-between style flosser. Panel D depicts reseeding the loop from the proximal opening, while the distal end is closed with an absorbable suture; arrowhead marks absorbable suture. Panel E depicts the resultant anatomy of the end to side loop; dashed white outline denotes loop structure.

FIG. 5 depicts an embodiment of an acute transmural intestinal damage model. Panel A depicts representative hematoxylin and cosin stained sections of healthy rat jejunum (left) and freshly damaged rat jejunum loop (right); scale bar=50 μm; n=3 for both groups. Panel B depicts representative scanning electron micrographs of healthy jejunum (left) and freshly damaged jejunum loop right); scale bar=100 μm. Epithelium is largely denuded with visible disruptions in the muscularis due to the chemical and mechanical damage model.

FIG. 6A-B depicts embodiments of survival associated with the mucosectomy procedure with a ten week post-operative endpoint. FIG. 6A depicts an embodiment of a Kaplan-Meier curve related to the mucosectomy procedure with a ten week post-operative endpoint. A total of 32 procedures were performed, shams/media reseeded n=3, fragmented HIO reseeding n=17 (11) and fragmented enteroid reseeding n=12 (8). FIG. 6B depicts an embodiment of a representative time of harvest image of an end to side blind loop ten weeks post-operation.

FIG. 7A-B depict embodiments of early loop engraftment of HIOs that regenerate the stem cell niche over time. FIG. 7A depicts an embodiment of representative images of loops after seven days reseeded with media alone or fragment HIOs stained for a human specific marker (KU80) and nuclei (hematoxylin) (upper panels) or with an epithelial marker (CDH1), a marker of proliferation (MK167), and nuclei (DAPI) (lower panels). Engrafted human contributions were exclusive to loops reseeded with dissociated HIOs. n=4 for both groups. FIG. 7B depicts an embodiment of representative images of control human jejunum and reseeded loops after ten weeks stained for a marker of proliferation (MKI67), an epithelial marker (CDH1), a surrogate marker of stem cell activity (OLFM4), and a telocyte marker (F3). n=3 for both groups. All scale bars=100 μm.

FIG. 8A-B depict embodiments of images showing that regional identity of engrafted HIOs is maintained. FIG. 8A depicts an embodiment of representative images of human jejunum and reseeded loops stained for a proximal intestinal epithelial transcription factor (GATA4) and epithelium (CDH1) (left panels), Paneth cell marker (DEFA5) (middle panels), and an enzyme involved in carbohydrate digestion (SI) (right panels). FIG. 8B depicts an embodiment of representative images of human colon and reseeded loops stained for a DNA binding protein present in the distal ileum and colon (SATB2), a colonocyte marker (MS4A12), and a colonic mucin (MUC5B). All scale bars=100 μm; n=3 for all groups.

FIG. 9A-H depict embodiments of data showing that neo-epithelia of reseeded loops are responsive to chemical stimuli. FIG. 9A depicts an embodiment of representative brightfield (left panel) and live GFP (right panel) images of healthy rat jejunum (proximal to the loop) mounted in a slider for an Ussing assay; dashed circle=opening of sider. FIG. 9B depicts an embodiment of representative brightfield (left panel) and live GFP (right panel) images of reseeded loop amounted in a slider for an Ussing assay; dashed circle=opening of slider. GFP expression verifies successful reseeding of the segment and origin of cells. FIG. 9C-D depict embodiments of representative time course of short circuit currents (I_{sc}) measured in Ussing chamber experiments using healthy jejunum (FIG. 9C) and GFP+ reseeded loops (FIG. 9D). FIG. 9E depicts an embodiment of graphs of calculated changes in I_{sc} in response to 10 μM forskolin, 100 μM IBMX, and 100 μM bumetanide. FIG. 9F depicts an embodiment of a graph of baseline transepithelial electrical resistance of healthy rat jejunum and GFP+ reseeded loops. FIG. 9G depicts an embodiment of a graph of FITC-dextran permeability of healthy rat jejunum and GFP+ reseeded loops over three hours (the upper line being the Loop). FIG. 9H depicts an embodiment of calculated FITC flux from the graph of FIG. 9G. Wilcoxon Signed-Ranks tests were used to determine statistical significance between groups in FIGS. 9E, 9F, and 9H. n=5 for both groups.

FIG. 10A-D depict embodiments of in vitro HIO and enteroid fragmentation and determination of cell counts for reseeding. FIG. 10A depicts an embodiment of representative brightfield and GFP images of an intact HIO (left panels) and HIO fragments (right panels) produced by shearing through a series of needles, ending at 25 G. Scale bars=500 μm. FIG. 10B depicts the same as FIG. 10A but using enteroids instead of HIO. FIG. 10C depicts an embodiment of representative brightfield image acquired during automated cell counter quantification of dissociated HIOs or enteroids. FIG. 10D depicts an embodiment of a graph of the average number of cells per HIO or enteroid as calculated from single nuclei preparations; n=3 HIO and n=4 enteroid preparations.

FIG. 11A-B depict embodiments of HIOs did not demonstrate biodistribution within their hosts. FIG. 11A depicts an embodiment of representative images of tissues 10 weeks post-operatively stained for a human specific marker (KU80, brown) and nuclei (hematoxylin, blue). In a transplanted HIO, there are primarily KU80+ cells, however in various organs of rats undergoing a seeded mucosectomy procedure, KU80+ cells were not observed. Scale bar=100 μm. FIG. 11B depicts an embodiment of a dot plot of Ct values from Alu PCR on organs collected from rats, as depicted in FIG. 11A. Dotted line indicates Ct value threshold for a positive signal (presence of human cells). An open circle indicates an undetermined value, or Ct of greater than 40. No data points fell within the range of human cellular presence. n=3 per group.

### DETAILED DESCRIPTION

Disclosed herein are clinically relevant methods and protocols for intestinal reconstitution and injury repair using dissociated cells from intestinal organoids or colonic organoids generated from pluripotent stem cells. Dissociated cells from intestinal organoids or colonic organoids, such as human intestinal organoids (HIOs) and human colonic organoids (HCOs) derived from human pluripotent stem cells, can engraft and contribute to regeneration within a damaged loop of host bowel in vivo and reconstitute both the mucosa and muscularis. From a clinical standpoint, the data is exciting as new treatment strategies for chronic, refractive intestinal diseases will require transmural regenerative potential. As disclosed herein, a substantial engraftment of organ surface area is achieved within ten weeks (an average engraftment of 16.93% by surface area after ten weeks, compared to only 1.68% when using enteroids), indicating that the intestinal organoid or colonic organoid seeding material is not outcompeted or washed out over time and more efficient than epithelial only seeding material.

In a previous study, Yui et al. report an engraftment/expansion rate of donor cells in colitic mice as 0.02% cells or about 100 cells per mouse four weeks post-transplantation when using enteroids (i.e. organoid-like structures derived from adult intestinal tissue comprising only epithelium and no mesenchyme). Several similar reports also demonstrate the ability of enteroids to contribute to intestinal and colonic healing, but efficiencies are rarely reported. Most recently, Sugimoto et al. demonstrated ileal enteroid engraftment within the mouse colon.

The disclosure herein is the first report of using multilineage seeding material, including both epithelial and mesenchymal, to achieve in vivo intestinal repair as a potential cell therapy. The methods here allow for an epithelial stem cell niche to be generated exclusively by the seeding material in areas where the native tissue has failed or been damaged. Upon incorporation, the epithelial stem cell compartment reemerged by ten weeks and the regional identity of the engrafted HIO fragments were retained. Loop neo-epithelia were functional and responsive to chemical stimuli with appropriate barrier integrity as observed with ex vivo physiologic Ussing chamber assays. Using pluripotent stem cell derived organoids that represent more diverse and specific regions of the gut, the platform can be extended to additional areas of the gastrointestinal tract, like the colon.

Pluripotent stem cell-derived organoid technologies, particularly for humans, have changed the landscape of tissue engineering over the last decade. The fundamental goal of tissue engineering lies within the ability of a generated material to functionally restore or improve damaged tissue or whole organs. Using an in vivo transmural injury model of the intestine, the therapeutic capacity of intestinal organoids was explored. HIOs generated de novo from hPSCs contain both epithelial and mesenchymal components. In a xenograft pre-clinical damage model, luminally derived dissociated HIOs were demonstrated to engraft and expand during the regenerative process. Not only was restitution of the mucosal layer observed, but significant incorporation was also observed throughout the muscularis. Further analysis revealed a reemergence of the epithelial stem cell/progenitor system for homeostatic renewal and retention of small bowel regionalization. Observed through ex vivo physiologic readings, neo-epithelium responded to chemical stimuli and its permeability was similar to healthy adjacent host bowel. The findings provide an exciting proof of concept for the therapeutic use of HIOs to treat chronic non-healing ulcerated intestinal injuries where fully restorative treatment options are currently lacking.

Two potential cell therapy sources for healing intestinal damage are enteroids and human intestinal organoids (HIOs). Enteroids are in vitro, epithelial-only structures that are derived from crypts isolated from patient intestine or transplanted HIOs. While they may contain all the differentiated epithelial cell subtypes, such as enterocytes, goblet cells, Paneth cells, and enteroendocrine cells, they lack the mesenchymal, neuronal, and immune compartments present in the human intestine. Some evidence indicates that enteroids can be used to replace damaged intestinal epithelium, however they cannot fully address transmural injuries. Unlike enteroids, HIOs are generated via stepwise differentiation of human pluripotent stem cells using the same small molecule growth factors that promote differentiation of fetal intestinal tissue in utero. HIOs contain both epithelial and mesenchymal cell types, which form into laminated structures upon transplantation. Utilizing an immunocompromised host as a bioreactor for engraftment and maturation, transplanted HIOs develop into structures reminiscent of human intestine, including a crypt/villus axis, vasculature, a muscularis mucosac and both the inner circular and outer longitudinal smooth muscle layers. The capacity of both enteroids and HIOs to regenerate damaged bowel in a preclinical rodent damage model was explored.

Methods of producing organoids or enteroids can be found in U.S. Pat. Nos. 9,719,068 and 10,174,289, and PCT Publications WO 2015/183920, WO 2016/061464, WO 2017/192997, WO 2018/085615, WO 2018/085622, WO 2018/085623, WO 2018/226267, WO 2018/106628, WO 2018/200481, WO 2018/191673, WO 2019/074793, WO 2019/126626, WO 2020/056158, WO 2020/023245, WO 2020/160371, WO 2020/243613, WO 2021/030373, each of which is hereby expressly incorporated by reference in its entirety.

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “intestinal organoid” as used herein has its plain and ordinary meaning as understood in light of the specification and refers to three-dimensional cellular structures that present many properties of the small intestine of an organism. In some embodiments, intestinal organoids relate to those derived from human cells and exhibit the properties of a human small intestine. However, intestinal organoids from other mammals are also encompassed. Intestinal organoids as used herein are derived from pluripotent stem cells (e.g. embryonic stem cells or induced pluripotent stem cells) or an intermediate thereof (e.g. definitive endoderm), where the process of differentiating pluripotent stem cells into definitive endoderm, then hindgut endoderm (which may be in the form of spheroids), and finally to an intestinal organoid results in a cellular structure that has the composition, structure, and function resembling a naturally developed intestine. A significant difference between the intestinal organoids used herein and enteroids, which are cellular structures derived from adult intestinal epithelium, and other so-called organoids produced from non-pluripotent adult intestinal stem cells, is that the intestinal organoids used herein contain both epithelium and mesenchyme. The mesenchyme performs an important supportive role for the epithelium, and greatly enhances the viability and robust function of the intestinal organoid. The intestinal organoids used herein may exhibit a lumen with epithelial villus-like involutions closely resembling normal intestine, and peristaltic behavior. As a result of the differentiation process from pluripotent stem cells, the intestinal organoids used herein also contain specialized intestinal cell types, including enterocytes, Goblet cells, Paneth cells, and enteroendocrine cells. References disclosing embodiments of intestinal organoids suitable for use herein include WO 2011/140441, WO 2016/061464, WO 2018/200481, WO 2020/160371, and WO 2021/030373, each of which are incorporated herein by reference in their entirety.

The term “colonic organoid” as used herein has its plain and ordinary meaning as understood in light of the specification and refers to three-dimensional cellular structures that present many properties of the colon of an organism. In some embodiments, colonic organoids relate to those derived from human cells and exhibit the properties of a human colon. However, colonic organoids from other mammals are also encompassed. Colonic organoids as used herein are derived from pluripotent stem cells (e.g. embryonic stem cells or induced pluripotent stem cells) or an intermediate thereof (e.g. definitive endoderm), where the process of differentiating pluripotent stem cells into definitive endoderm, then hindgut endoderm (which may be in the form of spheroids), and finally to a colonic organoid results in a structure that has the composition, structure, and function resembling a naturally developed colon. A significant difference between the colonic organoids used herein and colonoids, which are cellular structures derived from adult colon epithelium, and other so-called organoids produced from non-pluripotent adult colon stem cells, is that the colonic organoids used herein contain both epithelium and mesenchyme. The mesenchyme performs an important supportive role for the epithelium, and greatly enhances the viability and robust function of the colonic organoid. The colonic organoids used herein may exhibit a lumen with crypts but substantially free of villus-like structures. As a result of the differentiation process from pluripotent stem cells, the colonic organoids used herein also contain specialized colonic cell types, including a high number of Goblet cells (relative to intestinal organoids) and colonic enteroendocrine cells, but substantially free of Paneth cells. References disclosing embodiments of colonic organoids suitable for use herein include WO 2018/106628, which is incorporated herein by reference in their entirety.

The terms “fragmentation,” “fragmented,” “dissociation,” and “dissociated” as used herein have their plain and ordinary meanings as understood in light of the specification and refer to the partial or complete fragmentation or dissociation of an organoid or other three-dimensional multicellular structure to produce a population of single cells and viable multicellular structures, fragments, or clumps, without excessively shearing or damaging the cells such that that all or the majority of dissociated organoid comprises intact and healthy cells. Accordingly, “fragmented” and the like does not generally refer to, e.g., non-living subcellular components or fragments of single cells, such as liberated intracellular contents or non-living vesicles, although these components may be present in embodiments of fragmented organoid compositions by way of natural apoptosis of cells or unintended damage during dissociation of organoids. Fragmentation or dissociation of the organoid may be done in a variety of methods generally known in the art. The process of fragmentation or dissociation may be such that some of the resultant cells are found as small multi-cellular clumps/fragments rather than as single cells. The population of dissociated cells comprising multi-cellular clumps/fragments among single cells is contemplated for use herein. In some embodiments, the dissociated cell populations or compositions are present exclusively as multi-cellular clumps/fragments. In some embodiments, the dissociated cell populations or compositions are present exclusively as single cells without multi-cellular clumps/fragments. In some embodiments, the dissociated cell populations or compositions are predominantly (e.g. greater than 70%, 80%, or 90% of cells) multi-cellular clumps/fragments, with relatively few single cells. In some embodiments, the dissociated cell populations or compositions are present as a mixture of single cells and multi-cellular clumps/fragments.

The term “enzymatic dissociation” as used herein has its plain and ordinary meaning as understood in light of the specification and refers to fragmentation or dissociation of an organoid or other three-dimensional multicellular structure using the catalytic activity of one or more enzymes. A process generally well known in the art, enzymatic dissociation typically involves the use of proteolytic enzymes (e.g. trypsin), or enzymes specific for other molecules (e.g. hyaluronidase) involved in adherence to surface or intercellular bonds.

The term “mechanical dissociation” as used herein has its plain and ordinary meaning as understood in light of the specification and refers to fragmentation or dissociation of an organoid or other three-dimensional multicellular structure using a mechanical force. A process generally well known in the art, mechanical dissociation may be accomplished, for example, through trituration through narrow bore channels, where the channels may be in the form of pipettes, needles, microfluidic channels, or the like.

The terms “multi-cellular clump”, “clump of cells”, “multi-cellular fragments”, “multi-cellular organoid fragments” and the like as used herein have their plain and ordinary meanings as understood in light of the specification and refer to cells that are collected through adherent forces such as naturally produced extracellular matrices, where generally these collections of cells move as a single entity (e.g. within an aqueous suspension). These multi-cellular clumps or organoid fragments, as described herein, are generated through dissociation of organoids and/or enteroids through classical enzymatic and/or mechanical dissociation means. Accordingly, a person skilled in the art would be able to determine the approximate parameters (e.g., number of cells per clump/fragment, size, diameter, volume, largest dimension, etc.) of the multi-cellular clumps/fragments generated through these dissociation means. For example, the bore size of a narrow bore channel used for mechanical dissociation may have an effect on the resultant size of the clumps/fragments. These parameters may be quantified through conventional methods, such as microscopy or flow cytometry. As also discussed herein, these “multi-cellular fragments” refer to groups of living cells derived from the fragmentation or differentiation of larger three-dimensional cellular structures such as organoids, and not referring to subcellular components, although these subcellular components may be present in a composition due to the method of fragmentation or dissociation of the larger three-dimensional cellular structure.

As applied to the disclosure herein, the multi-cellular clumps/fragments produced from the dissociation of organoids and/or enteroids may be quantified in terms of number of cells per clump/fragment. In some embodiments, the multi-cellular clumps/fragments may comprise a number of cells that is, is about, is at least, is at least about, is not more than, or is not more than about, 10², 10³, 10⁴, 10⁵, or 10⁶ cells, or any number of cells within a range defined by any two of the aforementioned number of cells, for example, 10² to 10⁶ cells, 10² to 10⁴ cells, 10⁴ to 10⁶ cells, or 10³ to 10⁵ cells. As applied to the disclosed herein in some embodiments, the multi-cellular clumps/fragments produced from the dissociation of organoids and/or enteroids may be quantified in terms of the approximate diameter (or more generally, length of the greatest dimension for irregularly celled clumps/fragments) of the multi-cellular clumps/fragments. In some embodiments, the multi-cellular clumps/fragments may have an approximate diameter and/or greatest dimension that is, is about, is at least, is at least about, is not more than, or is not more than about, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 μm in diameter (or length of greatest dimension), or any diameter (or length of greatest dimension) between a range defined by any two of the aforementioned lengths, for example, 100-400 μm, 100-250 μm, 150-300 μm, 200-400 μm, or 200-250 μm.

The term “mucosa” as used herein has its plain and ordinary meaning as understood in light of the specification and refers to the most inner layer of the gastrointestinal tract. The epithelium is the most inner layer of the mucosa, and is where epithelial cells and other specialized cells such as Goblet cells are found. The epithelium also forms the villi structure of the intestine. The epithelium is surrounded by connective tissue called the lamina propria, and a thin layer of smooth muscle.

The term “muscularis” as used herein has its plain and ordinary meaning as understood in light of the specification and refers to the muscularis propria of the gastrointestinal tract. The muscularis regulates peristaltic behavior of the intestine and colon, and originates from the mesenchymal layer of the nascent gut tube during development.

The term “regionality” as used herein has its plain and ordinary meaning as understood in light of the specification and refers to the qualities and features that distinguish one cell type from another. In the context of intestine and colon (and other gastrointestinal organs), both organs originate from the same definitive endoderm but early specification results in the proper development and differentiation of the two organs and constituent cells commensurate with their function. Consequently, intestinal tissue exhibits a different regionality than colon tissue. As shown herein, intestinal and colonic organoids used for engraftment in an intestinal injury model retain their respective qualities even after integration into the cell layers of a different organ (e.g. intestinal organoid into host colon tissue or colonic organoid into host intestinal tissue).

The term “intestinal barrier” as used herein has its plain and ordinary meaning as understood in light of the specification and refers to the cellular and mucosal barrier that separates the intraluminal contents of the gastrointestinal tract from the surrounding tissue and circulatory system, while still permitting nutrient exchange. This barrier is mediated by the intracellular junctions between the cells of the epithelium. During intestinal damage, this barrier can be disrupted, resulting in abnormal function of the intestine, passage of potentially pathogenic microorganisms or antigens into the body, and leaking of blood and molecules into the lumen.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation typically suits the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The disclosure herein generally uses affirmative language to describe the numerous embodiments. The disclosure also includes embodiments in which subject matter is excluded, in full or in part, such as substances or materials, method steps and conditions, protocols, or procedures.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. In theory, induced pluripotent stem cells can be reprogrammed from any type of adult somatic cell. Typically, somatic cells that are relatively easy to isolate are used to reprogram into iPSCs. For example, dermal fibroblasts can be isolated from a subject through a skin biopsy, and peripheral blood mononuclear cells can be isolated from the peripheral blood of a subject. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx 15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCO1 (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “definitive endoderm” or “DE” as used herein has its plain and ordinary meaning as understood in light of the specification and refers to the developmental cell type that gives rise to the gut tube and resultant gastrointestinal organs, including the esophagus, stomach, small intestine, colon, liver, and pancreas. The anterior DE forms the foregut and its associated organs, including the liver and pancreas, and the posterior DE forms the midgut and hindgut, which forms the small and large intestines and parts of the genitourinary system. Markers of DE include SOX17 and FOXA2. During development, the Wnt and FGF signaling pathways establish regionalization between anterior and posterior patterning of the DE. Pluripotent stem cells can be differentiated into definitive endoderm by culturing the pluripotent stem cells with one or more transforming growth factor β (TGFβ) growth factor family members, such as Activin A, Activin B, or Nodal. As explored previously, the definitive endoderm can be differentiated into three-dimensional organoid structures resembling downstream gastrointestinal organs.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

### Intestinal and Colonic Organoids and Methods of Making

The intestinal and colonic organoids disclosed herein are produced by a differentiation process from pluripotent stem cells (such as embryonic stem cells or induced pluripotent stem cells) or an intermediate thereof (such as definitive endoderm), and comprise epithelial cell types and mesenchymal cell types, along with intestinal or colonic specialized cell types. Exemplary methods for making intestinal and colonic organoids can be found in U.S. Pat. Nos. 9,719,068 and 10,174,289, and PCT Publications WO 2016/061464, WO 2018/106628, WO 2018/200481, WO 2019/126626, WO 2020/160371, WO 2021/030373, each of which is hereby expressly incorporated by reference in its entirety.

In some embodiments, intestinal and colonic organoids are differentiated through the culture of definitive endoderm cells. These definitive endoderm cells can be differentiated from pluripotent cells by contacting the definitive endoderm with the Nodal, Activin, and/or BMP subgroups of the TGFβ superfamily of growth factors. In some embodiments, the pluripotent stem cells are contacted with Nodal, Activin A, Activin B, BMP4, or any combination thereof, to differentiate the pluripotent stem cells to definitive endoderm. In some embodiments, the pluripotent stem cells are contacted with Activin A to differentiate the pluripotent stem cells to definitive endoderm.

Definitive endoderm can further be subjected to FGF/Wnt-induced posterior endoderm patterning to direct hindgut specification.

In some embodiments, to produce intestinal and colonic organoids, definitive endoderm is first contacted with a Wnt signaling pathway activator and an FGF signaling pathway activator to posteriorize the definitive endoderm to hindgut endoderm. During this culture process, hindgut endoderm grows as monolayer but also spontaneously buds off as clumps of cells called hindgut spheroids in suspension. In some embodiments, the Wnt signaling pathway activator comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, or Wnt16, or any combination thereof. In some embodiments, the Wnt signaling pathway activator is Wnt3a. In some embodiments, the Wnt signaling pathway activator comprises a glycogen synthase kinase-3 (GSK3) inhibitor, which acts as a Wnt signaling pathway activator. In some embodiments, the GSK3 inhibitor is CHIR99021. In some embodiments, the FGF signaling pathway activator comprises FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, FGF23, or any combination thereof. In some embodiments, the FGF signaling pathway activator is FGF4. The hindgut endoderm and hindgut spheroids produced comprise CDX2+ polarized epithelium surrounded by CDX2+ mesenchyme, and lack Alb and Pdx1, which denote foregut endoderm.

Following formation of hindgut endoderm, or hindgut spheroids, which can be manipulated in suspension and embedded in a basement membrane matrix (e.g. Matrigel) for three-dimension culture, the BMP signaling pathway regulates formation of distinct regional types of intestine. Inhibition of BMP signaling after the hindgut stage promotes a proximal intestinal fate (duodenum/jejunum). Activation of BMP signaling after the hindgut stage promotes a more distal intestinal cell fate (cecum/colon). In some embodiments, the hindgut endoderm is contacted with a BMP signaling pathway activator to differentiate the hindgut endoderm into an intestinal organoid. In some embodiments, the hindgut endoderm is contacted with a BMP signaling pathway inhibitor to differentiate the hindgut endoderm into a colonic organoid. In some embodiments, the BMP signaling pathway activator comprises BMP1, BMP2, BMP3, BMP4, BMP5, BMP6, BMP7, BMP8a, BMP8b, BMP10, BMP11, BMP15, IDE1, or IDE2, or any combination thereof. In some embodiments, the BMP signaling pathway activator comprises BMP2. In some embodiments, the BMP signaling pathway inhibitor comprises Noggin, RepSox, LY364947, LDN193189, or SB431542, or any combination thereof. In some embodiments, the BMP signaling pathway inhibitor comprises Noggin.

### Dissociated Organoid Compositions

Disclosed herein are cell suspensions comprising a dissociated cell population comprising epithelial cell types and mesenchymal cell types. In some embodiments, the dissociated cell population is dissociated from intestinal and/or colonic organoids, where the intestinal and/or colonic organoids comprise epithelial cell types and mesenchymal cell types. In some embodiments, the intestinal and/or colonic organoids have been derived from precursor cells selected from embryonic stem cells, induced pluripotent stem cells, and definitive endoderm cells. In some embodiments, the cell suspension or the intestinal and/or colonic organoids are allogeneic to a subject. In some embodiments, the cell suspension or the intestinal and/or colonic organoids have been derived from cells from a subject, and the intestinal and/or colonic organoids are autologous to the subject. In some embodiments, the cell suspension or the intestinal and/or colonic organoids have been derived from induced pluripotent stem cells derived from the cells isolated from the subject. In some embodiments, the dissociated cell population are prepared by enzymatic dissociation and/or mechanical dissociation of the intestinal and/or colonic organoids. In some embodiments, enzymatic dissociation comprises dissociating the intestinal and/or colonic organoids with trypsin, chymotrypsin, collagenase, papain, hyaluronidase, elastase, thermolysin, neutral protease, or any combination thereof. In some embodiments, mechanical dissociation comprises passing the intestinal and/or colonic organoids through successively narrower bore channels. In some embodiments, the dissociated cell population comprises MKI67+ proliferative cells. In some embodiments, the percentage of cells in the dissociated cell population that are mesenchymal cell types is, is about, is at least, is at least about, is not more than, or is not more than about, 10%, 15%, 20%, 25%, 30%, 40%, 45%, 50%, 55%, 60%, 65%, 70%, 75%, 80%, 85%, 90% or 95%, or a percentage within a range defined by any two of the aforementioned percentages, for example, 10-50%, 40-80%, 70-95%, 85-95%, or 30-95%. In some embodiments, the percentage of cells in the dissociated cell population that are mesenchymal cell types is, is about, is at least, is at least about, is not more than, or is not more than about, 85%, 86%, 87%, 88%, 89%, 90%, 91%, 92%, 93%, 94%, or 95%, or any percentage within a range defined by any two of the aforementioned percentages, for example, 85-95%, 85-90%, 90-95%, or 88-92%. In some embodiments, the remaining percentage of cells in the dissociated cell population is made up of epithelial cell types. In some embodiments, the percentage of cells in the dissociated cell population that are epithelial cell types is, is about, is at least, is at least about, is not more than, or is not more than about, than 0%, 5%, 10%, 15%, 20%, 25%, 30%, 35%, 40%, 45%, 50%, 55%, 60%, 65%, 70%, or 75%, or a percentage within a range defined by any two of the aforementioned percentages, for example, 0-75%, 0-25%, 0-15%, 5-25%, 5-15%, 10-50%, or 50-75%. In some embodiments, the percentage of cells in the dissociated cell population that are epithelial cell types is, is about, is at least, is at least about, is not more than, or is not more than about, 5%, 6%, 7%, 8%, 9%, 10%, 11%, 12%, 13%, 14%, or 15%, or any percentage within a range defined by any two of the aforementioned percentages, for example, 5-15%, 5-10%, 10-15%, or 8-12%. In some embodiments, the remaining percentage of cells in the dissociated cell population is made up of mesenchymal cell types.

In some embodiments, the concentration of the dissociated cell population in the cell suspension is, is about, is at least, is at least about, is not more than, or is not more than about, 10⁵, 10⁶, 10⁷, 10⁸, 10⁹, 10¹⁰, or 10¹¹ cells/mL, or any concentration of cells within a range defined by any two of the aforementioned concentrations, for example, 10⁵-10¹¹, 10⁵-10⁸, 10⁹-10¹¹ or 10⁶-10¹⁰ cells/mL. In some embodiments, the concentration of cells in the dissociated cell population that are mesenchymal cell types is, is about, is at least, is at least about, is not more than, or is not more than about, 10⁵, 10⁶, 10⁷, 10⁸, 10⁹, 10¹⁰, or 10¹¹ cells/mL, or any concentration of cells within a range defined by any two of the aforementioned concentrations, for example, 10⁵-10¹¹, 10⁵-10⁸, 10⁹-10¹¹ or 10⁶-10¹⁰ cells/mL. In some embodiments, the concentration of cells in the dissociated cell population that are epithelial cell types is, is about, is at least, is at least about, is not more than, or is not more than about, 10⁵, 10⁶, 10⁷, 10⁸, 10⁹, 10¹⁰, or 10¹¹ cells/mL, or any concentration of cells within a range defined by any two of the aforementioned concentrations, for example, 10⁵-10¹¹, 10⁵-10⁸, 10⁹-10¹¹ or 10⁶-10¹⁰ cells/mL.

In some embodiments, the dissociated cell population is made up of multi-cellular fragments at a percentage that is, is about, is at least, is at least about, is not more than, or is not more than about, 30%, 35%, 40%, 45%, 50%, 55%, 60%, 65%, 70%, 75%, 80%, 85%, 90%, 95%, 96%, 97%, 98%, 99%, or 100% of the total cells in the dissociated cell population, or any percentage within a range defined by any two of the aforementioned percentages, for example, 30-100%, 50-100%, 75-100%, 90-100%, 30-75%, or 50-95%. In some embodiments, the dissociated cell population is in the form of 30%, 35%, 40%, 45%, 50%, 55%, 60%, 65%, 70%, 75%, 80%, 85%, 90%, 95%, 96%, 97%, 98%, 99%, or 100% multi-cellular fragments. In some embodiments, the dissociated cell population is in the form of at least 30%, 35%, 40%, 45%, 50%, 55%, 60%, 65%, 70%, 75%, 80%, 85%, 90%, 95%, 96%, 97%, 98%, 99%, or 100% multi-cellular fragments.

In some embodiments, the mesenchymal cell types of the dissociated cell population express vimentin (VIM) and/or elastin microfibril interfacer 1 (EMILIN1). In some embodiments, the epithelial cell types of the dissociated cell population express E-cadherin (CDH1) and/or caudal type homeobox 2 (CDX2).

Also disclosed herein are pharmaceutical compositions comprising an effective amount of any of the cell suspensions and/or dissociated cell populations disclosed herein and at least one pharmaceutically acceptable carrier, excipient, or diluent.

### Methods of Use

In some embodiments, the intestinal and colonic organoids as disclosed herein or otherwise known in the art comprising epithelial cell types and mesenchymal cell types are used in the methods of repairing intestinal damage disclosed herein.

As provided herein, methods are directed to treating intestinal damage. Treatment of intestinal damage encompasses the restoration or amelioration of damaged intestinal tissue into a healthy state, or the slowing, inhibition, prevention, or abrogation of intestinal damage progression or incidence. Also encompassed is the improvement of one or more symptoms associated with intestinal damage. Herein, intestinal damage may refer to an injured state of intestinal tissue, which may, but not necessarily, be due to a mechanical and/or chemical insult, and may, but not necessarily, be associated with apoptotic and/or necrotic behavior of the intestinal tissue. Other forms of intestinal damage and symptoms thereof are also envisioned.

Disclosed herein are methods of treating intestinal damage of a subject in need thereof. In some embodiments, the methods comprise administering a dissociated cell population dissociated from intestinal and/or colonic organoid to the luminal wall of the intestine of the subject. In some embodiments, the dissociated cell population comprise epithelial cell types and mesenchymal cell types. In some embodiments, the mesenchymal cell types of the dissociated cell population express vimentin (VIM) and/or elastin microfibril interfacer 1 (EMILIN1). In some embodiments, the epithelial cell types of the dissociated cell population express E-cadherin (CDH1) and/or caudal type homeobox 2 (CDX2). In some embodiments, the percentage of cells in the dissociated cell population that are mesenchymal cell types is, is about, is at least, is at least about, is not more than, is not more than about, 10%, 15%, 20%, 25%, 30%, 40%, 45%, 50%, 55%, 60%, 65%, 70%, 75%, 80%, 85%, 90% or 95%, or a percentage within a range defined by any two of the preceding percentages, for example, 10-50%, 40-80%, 70-95%, 85%-95%, or 30-95%. In some embodiments, the percentage of cells in the dissociated cell population that are mesenchymal cell types is, is about, is at least, is at least about, is not more than, or is not more than about, 85%, 86%, 87%, 88%, 89%, 90%, 91%, 92%, 93%, 94%, or 95%, or any percentage within a range defined by any two of the aforementioned percentages, for example, 85-95%, 85-90%, 90-95%, or 88-92%. In some embodiments, the remaining percentage of cells in the dissociated cell population is made up of epithelial cell types. In some embodiments, the percentage of cells in the dissociated cell population that are epithelial cell types is, is about, is at least, is at least about, is not more than, or is not more than about, than 0%, 5%, 10%, 15%, 20%, 25%, 30%, 35%, 40%, 45%, 50%, 55%, 60%, 65%, 70%, or 75%, or a percentage within a range defined by any two of the aforementioned percentages, for example, 0-75%, 0-25%, 0-15%, 5-25%, 5-15%, 10-50%, or 50-75%. In some embodiments, the percentage of cells in the dissociated cell population that are epithelial cell types is, is about, is at least, is at least about, is not more than, or is not more than about, 5%, 6%, 7%, 8%, 9%, 10%, 11%, 12%, 13%, 14%, or 15%, or any percentage within a range defined by any two of the aforementioned percentages, for example, 5-15%, 5-10%, 10-15%, or 8-12%. In some embodiments, the remaining percentage of cells in the dissociated cell population is made up of mesenchymal cell types. In some embodiments, the dissociated cell population is administered as a cell suspension. In some embodiments, the concentration of the dissociated cell population in the cell suspension is, is about, is at least, is at least about, is not more than, or is not more than about, 10⁵, 10⁶, 10⁷, 10⁸, 10⁹, 10¹⁰, or 10¹¹ cells/mL, or any concentration of cells within a range defined by any two of the aforementioned concentrations, for example, 10⁵-10¹¹, 10⁵-10⁸, 10⁹-10¹¹ or 10⁶-10¹⁰ cells/mL. In some embodiments, the concentration of cells in the dissociated cell population that are mesenchymal cell types is, is about, is at least, is at least about, is not more than, or is not more than about, 10⁵, 10⁶, 10⁷, 10⁸, 10⁹, 10¹⁰, or 10¹¹ cells/mL, or any concentration of cells within a range defined by any two of the aforementioned concentrations, for example, 10⁵-10¹¹, 10⁵-10⁸, 10⁹-10¹¹ or 10⁶-10¹⁰ cells/mL. In some embodiments, the concentration of cells in the dissociated cell population that are epithelial cell types is, is about, is at least, is at least about, is not more than, or is not more than about, 10⁵, 10⁶, 10⁷, 10⁸, 10⁹, 10¹⁰, or 10¹¹ cells/mL, or any concentration of cells within a range defined by any two of the aforementioned concentrations, for example, 10⁵-10¹¹, 10⁵-10⁸, 10⁹-10¹¹ or 10⁶-10¹⁰ cells/mL. In some embodiments, the intestine of the subject as contemplated here comprises the small intestine and/or the colon. In some embodiments, administering the dissociated cell population to the luminal wall of the intestine of the subject comprises administering the cell population to a location of the lumen of the intestine affected by the intestinal damage. In some embodiments the location is directly adjacent to or near the intestine affected by the intestinal damage. In some embodiments, the dissociated cell population is administered to the surface of the luminal wall.

In some embodiments of any of the methods disclosed herein, the dissociated cell population is administered to the luminal wall of the intestine of the subject as a cell suspension. In some embodiments, the cell suspension is in an isotonic solution, such as saline or Ringer's lactate solution. In some embodiments, administering the cell population to the luminal wall of the intestine of the subject comprises administering the cell population by a non-invasive or minimally invasive process. In some embodiments, the cell population is administered by oroenteric catheter, nasoenteric catheter, or enema. In some embodiments, the cell population is administered by direct intraluminal injection.

In some embodiments of any of the methods disclosed herein, the intestinal and/or colonic organoids have been derived from precursor cells selected from embryonic stem cells, induced pluripotent stem cells, and definitive endoderm cells. In the intestinal and/or colonic organoids are allogeneic to the subject. In some embodiments, the intestinal and/or colonic organoids have been derived from cells isolated from the subject. In some embodiments, the intestinal and/or colonic organoids are autologous to the subject. In some embodiments, the intestinal and/or colonic organoids have been derived from induced pluripotent stem cells derived from the cells isolated from the subject. In some embodiments, the cells isolated from the subject may be any cells amenable for pluripotent reprogramming. Common cells amenable for pluripotent reprogramming that are used include dermal fibroblasts or peripheral blood mononuclear cells (PBMCs). In some embodiments, the cells isolated from the subject comprise dermal fibroblasts or PBMCs from the subject.

In some embodiments of any of the methods disclosed herein, the dissociated cell population dissociated from the intestinal and/or colonic organoids are prepared by enzymatic dissociation and/or mechanical dissociation of the intestinal and/or colonic organoids. In some embodiments, enzymatic dissociation comprises dissociating the intestinal and/or colonic organoids with trypsin, chymotrypsin, collagenase, papain, hyaluronidase, elastase, thermolysin, neutral protease, or any combination thereof, or any other cell dissociation enzyme or reagent otherwise known in the art. In some embodiments, mechanical dissociation comprises passing the intestinal and/or colonic organoids through successively narrower bore channels. In some embodiments, the channels may be needles, microfluidic channels, capillaries, or tubes. In some embodiments, the successively narrower bore channels comprise 18 gauge, 20 gauge, 21 gauge, 22 gauge, 23 gauge, or 25 gauge channels, or any combination thereof. In some embodiments, the successively narrower bore channels comprise 18 gauge, 20 gauge, 21 gauge, 22 gauge, 23 gauge, and 25 gauge channels, or a series of channels including or lacking any one, two, or three of the aforementioned gauge channels. In some embodiments, the successively narrower bore channels comprises and/or begin with an 18 gauge channel. In some embodiments, the successively narrower bore channels comprise a 20 gauge channel. In some embodiments, the successively narrower bore channels comprise and/or end with a 25 gauge channel. In some embodiments, the successively narrower bore channels comprise, consist essentially of, or consist of 18 gauge, 20 gauge, and 25 gauge channels.

In some embodiments of any of the methods disclosed herein, the dissociated cell population dissociated from the intestinal and/or colonic organoids is administered to the subject at a concentration that is, is about, is at least, is at least about, is not more than, or is not more than about, 50, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 cells per mm² of affected intestine surface area, or any amount of cells per mm² within a range defined by any two of the aforementioned values, for example, 50-10000 cells per mm², 50-5000 cells per mm², 50-1000 cells per mm², 5000-10000 cells per mm², 2000-8000 cells per mm², or 500-5000 cells per mm². The surface area of the affected intestine can be determined through conventional methods by a skilled person, for example, by measuring either the outer surface or inner surface of an intestine macroscopically, where the apparent increase in surface area due to villi projections can be either ignored or considered. In some embodiments, the dissociated cell population is administered to the subject for a number of times until an improvement in the intestinal damage is observed. In some embodiments, the dissociated cell population is administered to the subject for a number of times that is, is about, is at least, is at least about, is not more than, or is not more than about, 1, 2, 3, 4, 5, 6, 7, 8, 9, or 10 times.

In some embodiments of any of the methods disclosed herein, cells of the dissociated cell population integrate into the mucosa and muscularis of the intestine of the subject. In some embodiments, cells of the dissociated cell population integrated into the intestine of the subject maintain their intestinal and/or colonic regionality. In some embodiments, cells, or a subpopulation thereof, of the dissociated cell population integrated into the intestine of the subject differentiate into smooth muscle actin (SMA)-positive smooth muscle cell types. In some embodiments, the dissociated cell population comprises Marker of Proliferation KI67+ (MKI67+) proliferative cells that integrate into the intestine of the subject and promote healing of the intestinal damage. In some embodiments, the dissociated cell population improves the intestinal barrier in the intestine of the subject after administration to the subject. In some embodiments, the dissociated cell population promotes formation of an intact intestinal barrier in the intestine of the subject after administration to the subject. In some embodiments, the percentage of the dissociated cell population that integrates into the intestine of the subject is, is about, is at least, is at least about, 10%, 15%, 20%, 25%, 30%, 40%, 45%, 50%, 55%, 60%, 65%, 70%, 75%, 80%, 85%, 90% or 95%, or a range defined by any two of the preceding values, for example, 10-50%, 40-80%, 70-95%, or 30-95%. In some embodiments, cells of the dissociated cell population integrate into a surface area of the luminal wall of the intestine of the subject that is, is about, is at least, is at least about, is not more than, or is not more than about, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% of the total or damaged surface area of the luminal wall of the intestine of the subject, or any percentage of surface area within a range defined by any two of the aforementioned percentages, for example, 10% to 50% of the surface area, 10% to 25% of the surface area, 25% to 50% of the surface area, or 15% to 35% of the surface area. In some embodiments, a percentage of the repaired intestinal tissue made up of cells from the dissociated cell population is, is about, is at least, is at least about, is not more than, or is not more than about, 50%, 55%, 60%, 65%, 70%, 75%, 80%, 85%, 90%, 95%, or 99%, or any percentage within a range defined by any two of the aforementioned percentages, for example, 50-99%, 50-75%, 50-60%, 75-99%, or 65-85%.

In some embodiments of any of the methods disclosed herein, the methods disclosed herein are used to treat intestinal damage of a subject in need thereof. In some embodiments, the intestinal damage comprises intestinal ulceration. In some embodiments, intestinal ulceration may be associated with leaking of blood or protein into the lumen of the intestine. In some embodiments, the intestinal damage is chemical and/or mechanical. In some embodiments, the intestinal damage is associated with a gastrointestinal malady. In some embodiments, the gastrointestinal malady is selected from Crohn's disease, ulcerative colitis, enteropathies associated with non-steroidal anti-inflammatory drugs (NSAIDs) or other medications, radiation-induced enteropathies, and enteropathies associated with pathogenic infections, such as tuberculosis.

In some embodiments of any of the methods disclosed herein, the intestinal and/or colonic organoids or the dissociated cell population dissociated from the intestinal and/or colonic organoids are mammalian. In some embodiments, the intestinal and/or colonic organoids or the dissociated cell population dissociated from the intestinal and/or colonic organoids are human. In some embodiments, the subject is mammalian. In some embodiments, the subject is human.

In some embodiments of any of the methods disclosed herein, the methods further comprise producing intestinal and/or colonic organoids comprising epithelial cell types and mesenchymal cell types, and/or dissociating the intestinal and/or colonic organoids to produce the dissociated cell population comprising the epithelial cell types and mesenchymal cell types. In some embodiments, the dissociated cell population is made up of multi-cellular fragments at a percentage that is, is about, is at least, is at least about, is not more than, or is not more than about, 30%, 35%, 40%, 45%, 50%, 55%, 60%, 65%, 70%, 75%, 80%, 85%, 90%, 95%, 96%, 97%, 98%, 99%, or 100% of the total cells in the dissociated cell population, or any percentage within a range defined by any two of the aforementioned percentages, for example, 30-100%, 50-100%, 75-100%, 90-100%, 30-75%, or 50-95%. In some embodiments, the dissociated cell population is in the form of 30%, 35%, 40%, 45%, 50%, 55%, 60%, 65%, 70%, 75%, 80%, 85%, 90%, 95%, 96%, 97%, 98%, 99%, or 100% multi-cellular fragments. In some embodiments, the dissociated cell population is in the form of at least 30%, 35%, 40%, 45%, 50%, 55%, 60%, 65%, 70%, 75%, 80%, 85%, 90%, 95%, 96%, 97%, 98%, 99%, or 100% multi-cellular fragments. In some embodiments, the intestinal and/or colonic organoids are produced from pluripotent stem cells. The intestinal and/or colonic organoids may be produced according to methods disclosed herein, or otherwise known in the art.

Also disclosed herein are dissociated cell populations dissociated from intestinal and/or colonic organoids, and pharmaceutical compositions of the same, for use in a method of treating a gastrointestinal malady in a subject in need thereof as described herein.

Some embodiments described herein relate to pharmaceutical compositions that comprise, consist essentially of, or consist of an effective amount of a dissociated cell population or composition described herein and a pharmaceutically acceptable carrier, excipient, diluent, or combination thereof. A pharmaceutical composition described herein is suitable for human and/or veterinary applications.

Also disclosed herein are the cell suspensions or the pharmaceutical compositions provided herein for use in the treatment of intestinal damage.

### EXAMPLES

Some aspects of the embodiments discussed herein are disclosed in further detail in the following examples, which are not in any way intended to limit the scope of the present disclosure. Those in the art will appreciate that many other embodiments also fall within the scope of the disclosure, as it is described herein and in the claims.

### Example 1. Administration of Dissociated HIOs for Intestinal Injury Repair

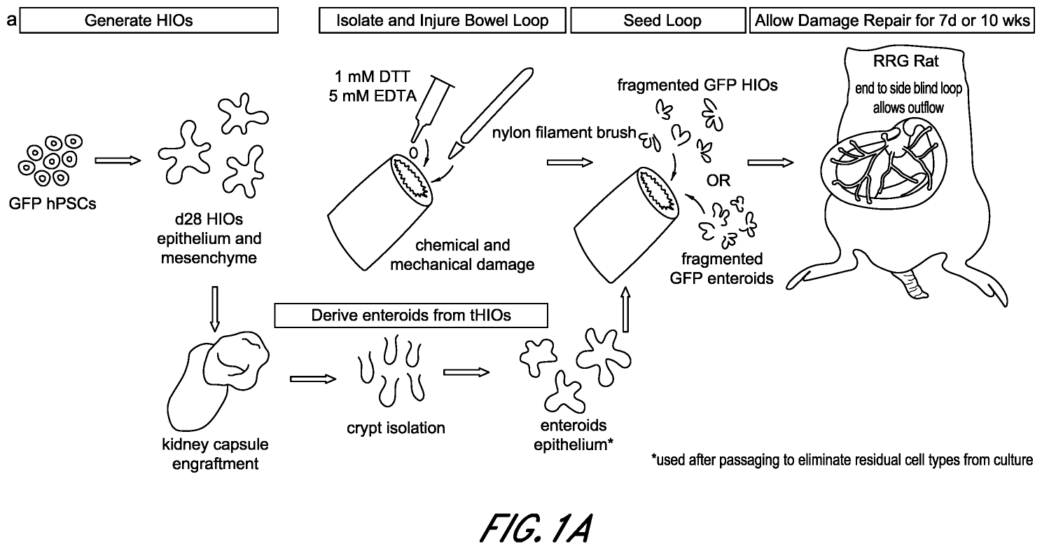

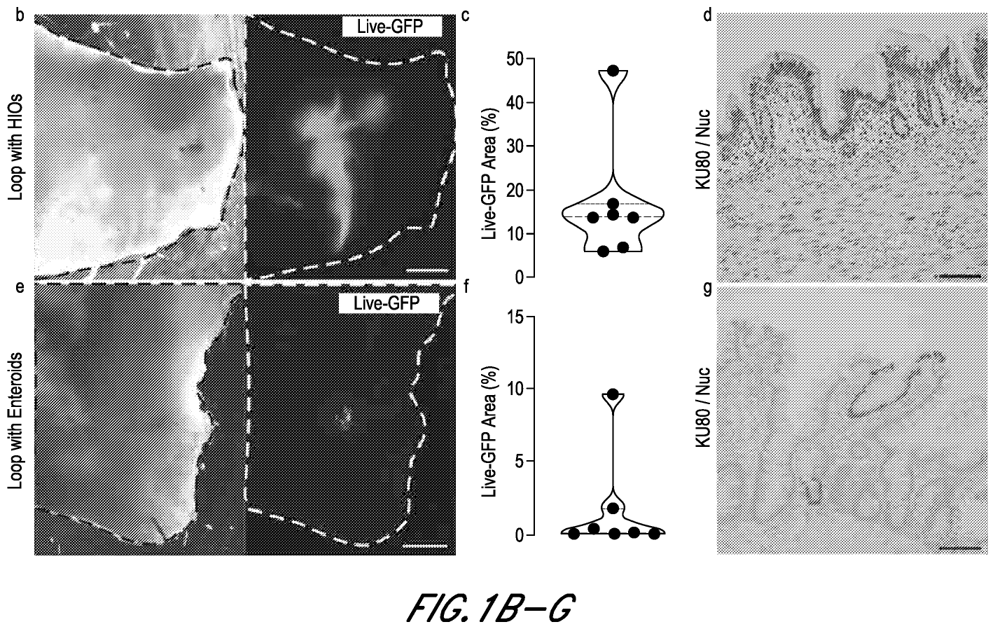

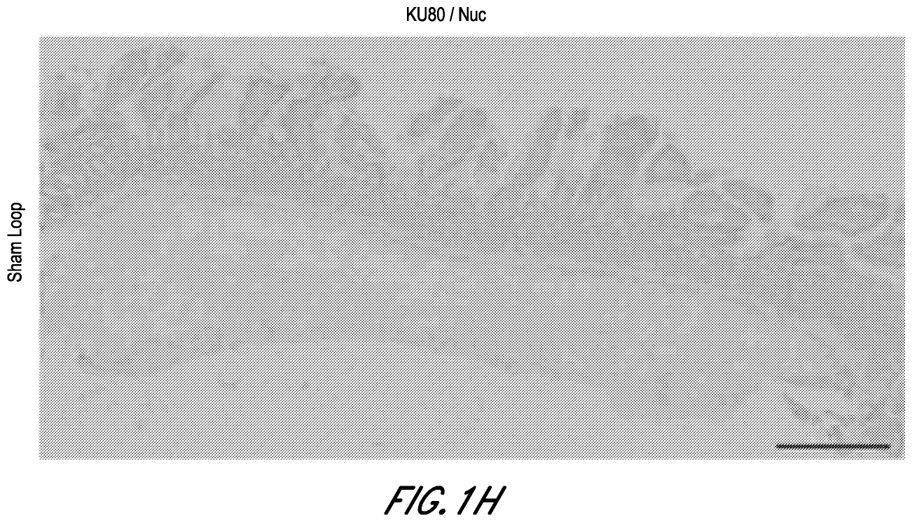

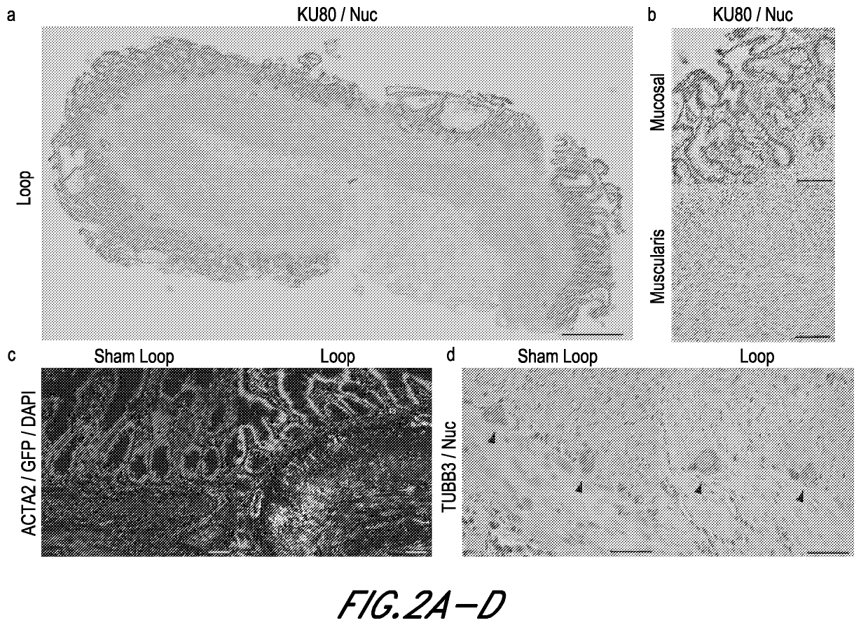

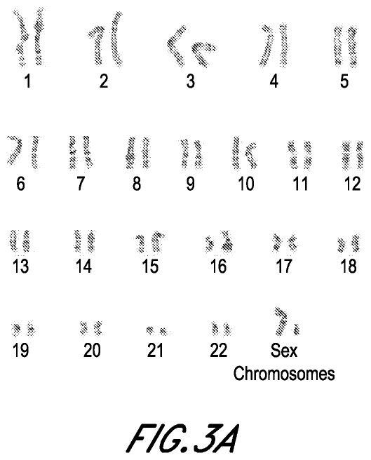

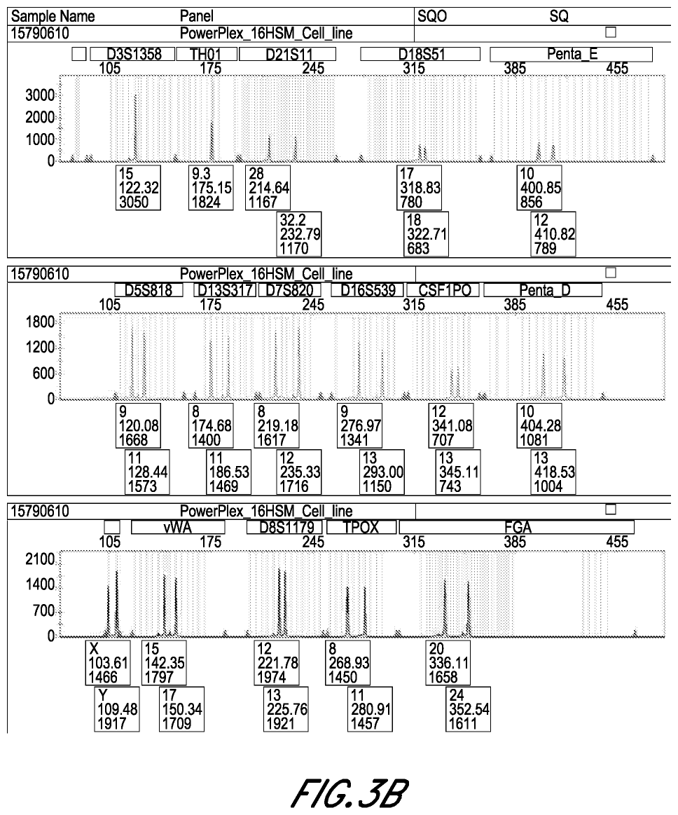

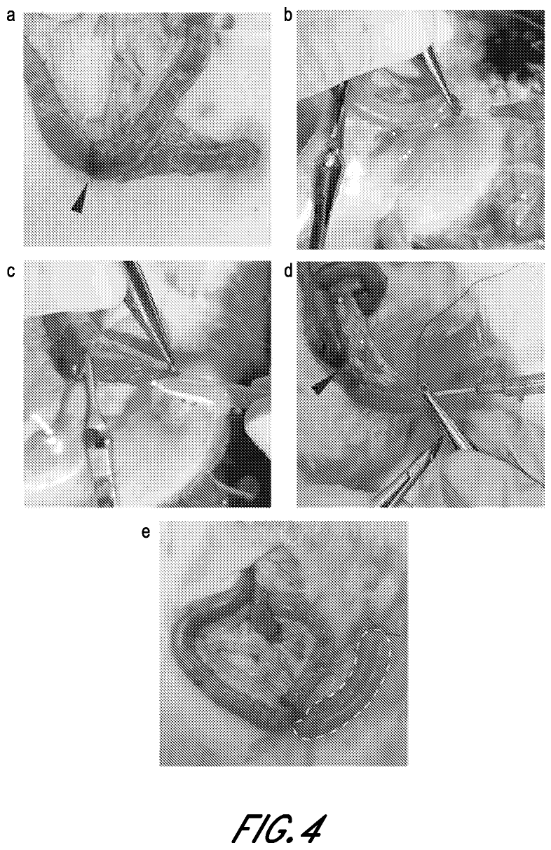

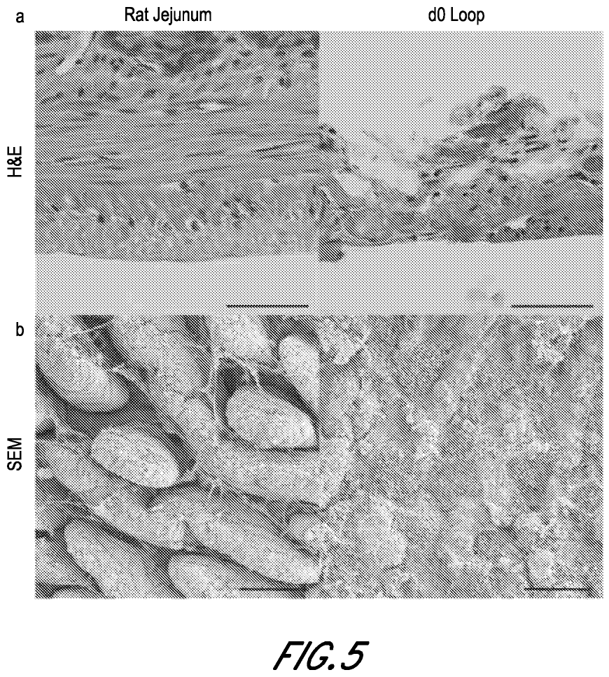

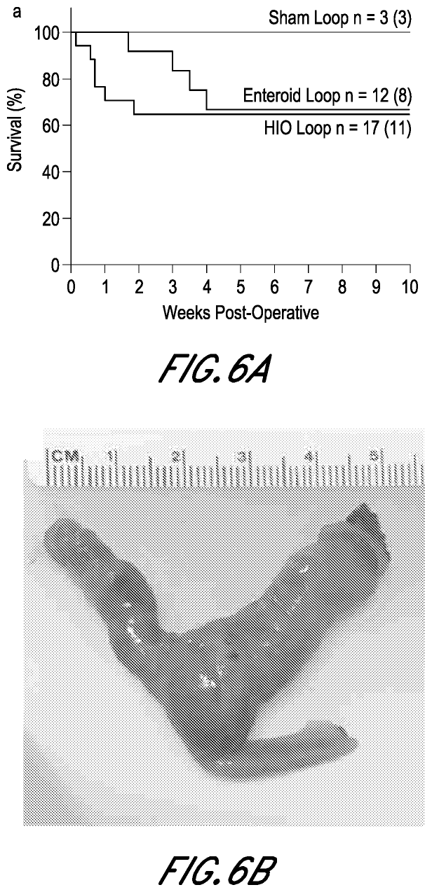

The generation of human intestinal organoids (HIOs) from pluripotent stem cells by manipulating growth factors to mimic early development and small intestinal specification were previously described (Spence et al. Nature. (2011) 470:105-109; McCracken et al. Nat. Protoc. (2011) 6:1920-1928; Watson et al. Nat. Med. (2014) 20:1310-1314). Upon differentiation, HIOs give rise not only to a complex epithelial layer containing all major cell types, but also a mesenchymal component that, when transplanted into an immunocompromised host, forms into a functional, laminated structure. Herein, the therapeutic potential of in vitro HIOs was assessed. An H1 embryonic stem cell line modified to constitutively express green fluorescent protein (GFP was used for case of downstream xenograft analysis in a pre-clinical model of damaged bowel. However, it is envisioned that other pluripotent stem cells, such as other embryonic stem cells and induced pluripotent stem cells) can be used. Constitutive expression of GFP in the HI cells were confirmed not to interfere with their karyotype (FIG. 3A). The modified cell line was characterized before use and determined to pass quality control metrics (FIG. 3A-3B). Immunocompromised Rag1 and II2g knockout (RRG) rats were utilized as host subjects to surgically create blind segments of distal small bowel that subsequently underwent chemical and mechanical injury between the immediate reseeding with dissociated HIOs or enteroids derived from transplanted HIOs (FIG. 1A). The surgical procedure performed resulted in a blind end-to-side “Y” segment, or loop, which was tied off with absorbable suture such that the segment could drain distally into the host intestinal tract (FIG. 4, panels A-E). Using this absorbable suture provided a brief window for HIO retention, epithelial restitution, and engraftment to occur. This was followed by a more physiologic state of intestinal drainage and luminal content exposure. The injury resulted in a segment of bowel that was predominantly denuded of epithelium with exposed mesenchyme, as observed histologically and via topographical micrographs, when compared to healthy rat jejunum (FIG. 5, panels A-B). Early mortality, within one week, was observed in some rats undergoing this procedure. In total, 68-70% of rats survived until the ten-week post-operative time point (FIG. 6A). This was attributed primarily to sepsis, as indicated by veterinary autopsy, likely due to the severity of the injury in the loop resulting in a failure to reestablish an effective epithelial barrier and translocation when exposed to luminal bacteria.

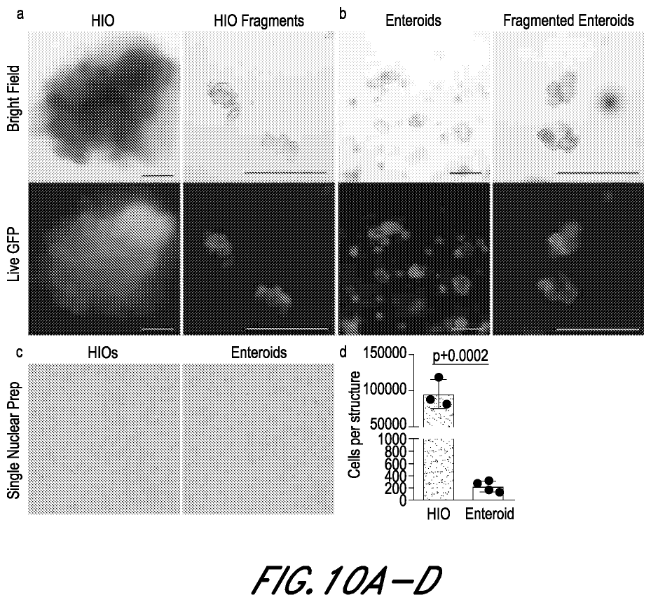

At the ten-week post-operative time point, loops were harvested and evaluated (FIG. 6B). In sham loops reseeded with media alone, healing was observed histologically. Immunohistochemistry for a human specific marker, Ku autoantigen, 80 kDa (KU80), was not detected in sham loops, indicating the absence of human cellular content (FIG. 1H). The observed healing illustrated that the small bowel has an intrinsic restorative capacity, and that epithelial restitution and reestablishment of barrier function can occur when provided time. However, in loops reseeded with dissociated GFP HIOs, regions expressing live-GFP were observed and comprised an average of 16.93% of the loop's surface area (FIG. 1B-C). Immunohistochemistry for KU80 revealed robust human cellular incorporation within the dissected GFP+ regions of reseeded loops (FIG. 1D). KU80+ cells were observed throughout the thickness of the intestine. In loops reseeded with fragmented GFP enteroids, regions expressing live-GFP were also observed, however they were significantly smaller and comprised only 1.68% of the loop's surface area on average (FIG. 1E-F). Again, human cellular incorporation was observed within the dissected GFP+ regions of enteroid fragment reseeded loops and the robustness was reduced in accordance with live-GFP expression observations (FIG. 1G). Here, KU80+ cell engraftment was restricted to the intestinal epithelium. In preforming these experiments, the cellular content being reseeded to the total number of cells was standardized, rather than discriminating between only the epithelial cell counts (FIG. 10A-D). After comparing the effectiveness of both HIOs and enteroids as a cell therapy source, only use of fragmented HIOs as the cell source was continued, because of their increased engraftment efficiency, expanded contribution to intestinal restitution across its full thickness and thus greater potential for clinical applications.

To further demonstrate the extent of human cellular contributions within loops reseeded with fragmented GFP HIOs, a tile scan was performed on a region of GFP+ loop stained for a human marker KU80 (FIG. 2A). Human cellular contributions were observed within the mucosa and the muscularis, which displayed proper circular and longitudinal muscle layer alignment (FIG. 2B). The humanization of the muscularis was further highlighted by immunostaining for both GFP and Actin Alpha 2, Smooth Muscle (ACTA2) in which the fibers presented as perpendicular in both sham (loops reseeded with media void of cellular content) and reseeded loops but GFP presence was only observed in reseeded loops (FIG. 2C). Clinically, this is an important feature as it broadens the application of potential cell therapy for conditions resulting in damage beyond the mucosa, such as non-healing ulcers and fistulizing Crohn's disease.

The effect of engraftment of HIOs to the intestinal injury model on host innervation and the enteric nervous system (ENS), which is responsible for a variety of functions including motility, was assessed. It was evaluated whether the myenteric plexus remained at the interface of the two newly formed human muscle layers, or if it had been displaced during the cellular expansion and remodeling process. This was an important facet to consider, as the effects on muscularis humanization on the ENS when using epithelium only or enteroid structures, rather than the HIOs herein containing additional cell types including mesenchyme, was unknown. Immunostaining for a pan-neuronal marker, Tubulin Beta 3 Class III (TUBB3) confirmed that the host innervation was not displaced after human cellular reconstitution suggesting that its function is also be preserved (FIG. 2D).

### Example 2. Reseeded HIOs Undergo Early Engraftment During Injury Healing and Maintain Regionality

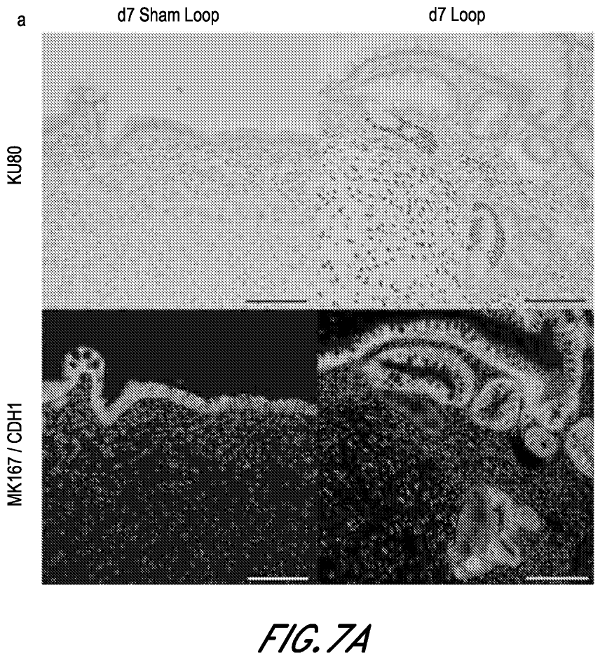

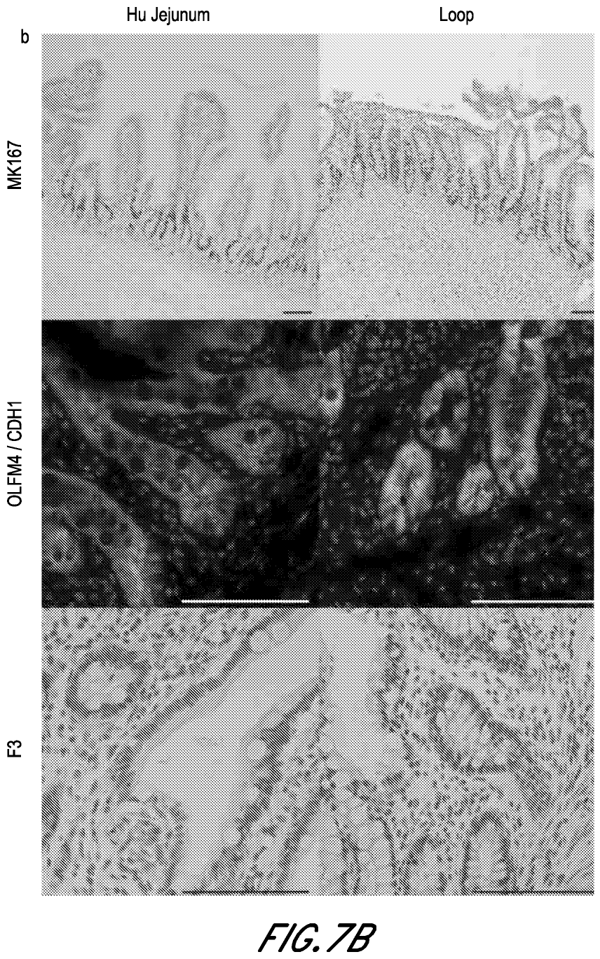

Having observed significant contributions by the reseeded HIOs to intestinal healing by ten weeks, additional experiments with a seven-day time point was performed to assess an earlier stage of engraftment. Immunohistochemistry for KU80 again revealed an absence of human cellular contributions in sham loops, while human cellular contributions were found in both the epithelium and mesenchyme of reseeded loops at day 7 (FIG. 7A). The level of epithelial structuration in sham loops was less extensive than that observed in reseeded loops. Furthermore, immunostaining for Marker of Proliferation KI67 (MKI67) revealed that in sham operated loops, the primary mechanism of healing at the seven-day time point was attributed to migration with few cells actively cycling and that were MKI67+ (FIG. 7B, left panels). However, in reseeded loops, MKI67+ cells were more broadly observed, indicating that HIO fragments played an active role during early engraftment and restitution of the bowel (FIG. 7B, right panels).

Next, it was determined by protein expression whether the engrafted HIOs' regenerative process resulted in later stage reconstruction of the epithelial stem/progenitor system for homeostatic renewal. Immunostaining for MKI67 in loops exhibited the stereotypical proliferative zonation similar to human jejunum controls (FIG. 7B). Expression of an antiapoptotic protein associated with stem and progenitor cells in the bowel, Olfactomedin 4 (OLFM4), was observed to be restricted within the crypt-like regions of loops, similar to the expression pattern observed in human jejunum controls (FIG. 7B). Subepithelial telocytes, an important source of niche signals to intestinal stem cells and epithelium, as marked by Coagulation Factor III, Tissue Factor (F3), were also observed to localize adjacent to the epithelium similarly within the loops and human jejunum controls (FIG. 7B). Taken together, these data support a reemergence of the epithelial stem cell compartment and niche in HIO derived neo-epithelium.

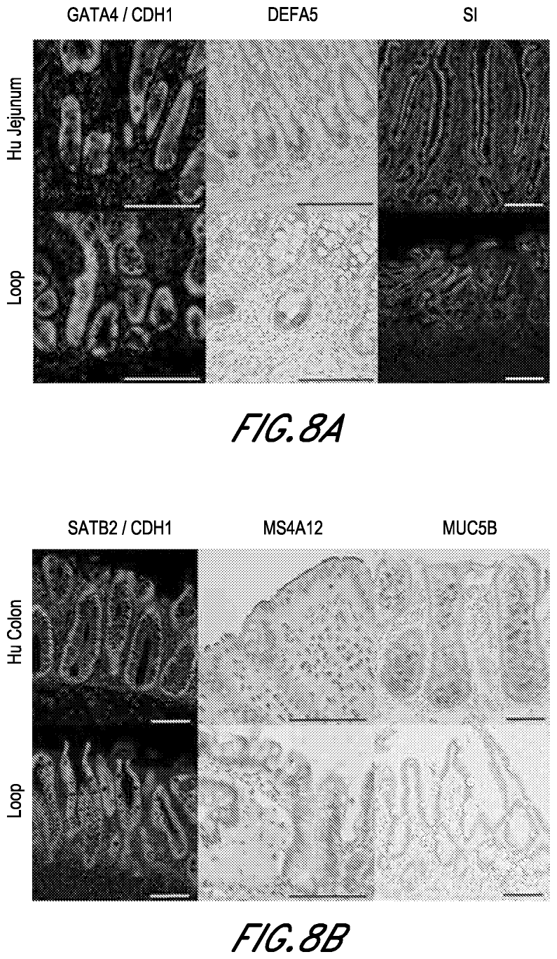

Subsequently, whether the small intestinal fate of the loop's HIO neo-epithelium was preserved was examined by protein expression. Immunostaining for a proximal regional marker, GATA binding protein 4 (GATA4), revealed positive expression in loops similar to human jejunum controls (FIG. 8A, left panels). Paneth cells, which are specialized secretory cells localized to small intestinal cryptos, as demonstrated by antimicrobial peptide Defensin Alpha 5 (DEFA5) expression, were detected, and localized to crypt-like structures in loops as observed in human jejunum controls (FIG. 8A, center panels). An enzyme involved in carbohydrate digestion within the small bowel, Sucrase Isomaltase (SI), was also observed and localized to the brush border in loops as also seen in human jejunum controls (FIG. 8A, right panels). As further validation, the expression of proteins restricted to the more distal small bowel and colon were also investigated. Immunostaining for a distal regional marker, DNA binding protein SATB Homeobox 2 (SATB2) was not observed in loops but found present throughout the epithelium of human colon controls (FIG. 8B, left panels). Membrane Spanning 4-Domains A12 (MS4A12), a calcium channel primarily localized in the apical membrane of colonocytes, was not observed in loops, but present throughout the epithelium of human colon controls (FIG. 8B, center panels). Mucin 5B (MUC5B), a gel forming mucus throughout the colon, was also absent from loops, but observed in Goblet cells of human colon controls (FIG. 8B, right panels). Taken together, positive protein expression of proximal intestinal markers along with the absence of distal markers suggest that the regionality of the loop's HIO neo-epithelium was maintained during the engraftment and expansion of the HIO fragments.

### Example 3. Reseeded HIOs Engraftment Form Intact Epithelial Barriers

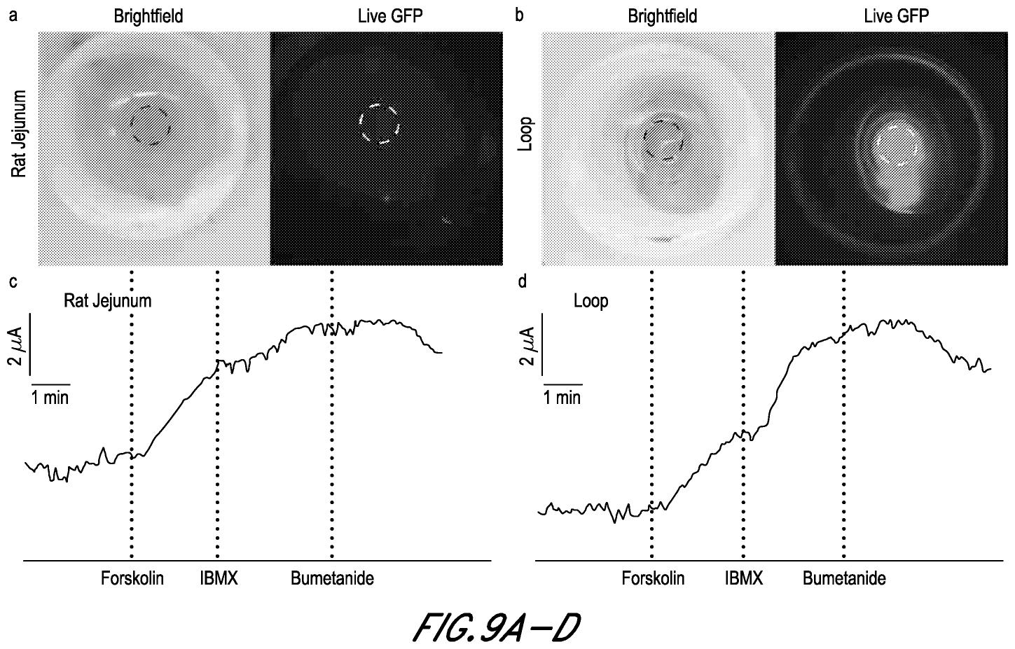

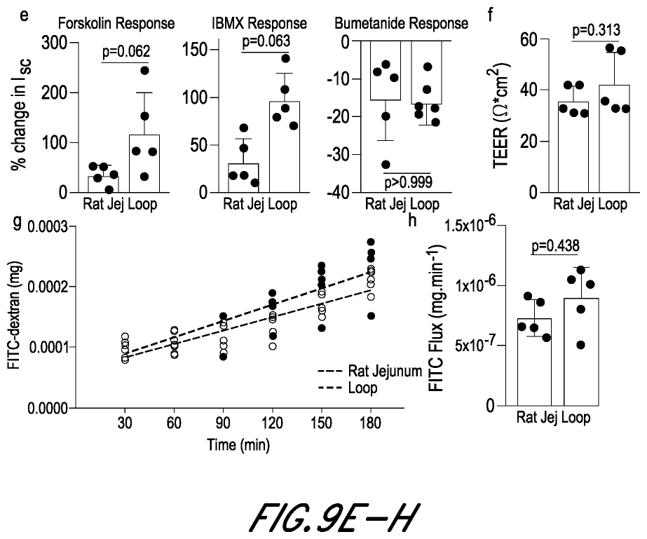

To gain functional insight, Ussing chamber assays were performed ten weeks postoperatively on the epitheliums of live-GFP expressing loop regions and healthy rat jejunum (proximal to the anastomosis site and outside the loop) (FIG. 9A-B). Epitheliums were exposed to a series of chemical stimuli while electrophysical properties were continuously recorded. Both loop and rat jejunum epithelia were responsive to challenges with forskolin, 3-isobutyl-1-methylxanthine (IBMX) and bumetanide as observed in real time recordings (FIG. 9C-D). Changes in short circuit current (I_{sc}), a reflection of active ion transport, were consistent with the known method of action of each compound applied for both groups. This demonstrated that appropriate cyclic adenosine monophosphate regulation and function of ion channels in the neo-epithelia was occurring. Furthermore, live-GFP expressing regions, the neo-epithelia demonstrated a higher sensitivity to both forskolin and IBMX when compared to healthy rat jejunum. Baseline readings of transepithelial electrical resistance (TEER) of rat jejunum were measured and observed within their established range (FIG. 9F). Resistance values without the muscularis and serosal layers have been previously documented between 20 and 45 Ω*cm² for rat small intestine. In the case of adult human small intestine, TEER values have been previously documents between 50 and 100 Ω*cm². While some of the loop TEER values fall within the established TEER range for adult human small intestine, others were observed to be lower. This difference may be associated with the known fetal state of the HIOs used for engraftment or a result of human epithelial interactions with host luminal content.

To further assess the integrity and stability of the loop neo-epithelium, paracellular permeability was evaluated. Fluorescein isothiocyanate (FITC)-dextran was added to the apical chamber of each specimen and samples were collected from the basolateral chamber every 30 minutes for evaluation of fluorescence intensity over time (FIG. 9G). From this dataset, FITC-dextran flux values were calculated and no difference between groups were observed (FIG. 9H). This indicates that loop neo-epithelium established a sufficient barrier during the healing process.

### Example 4. Biodistribution and Potential Expansion of HI Cells

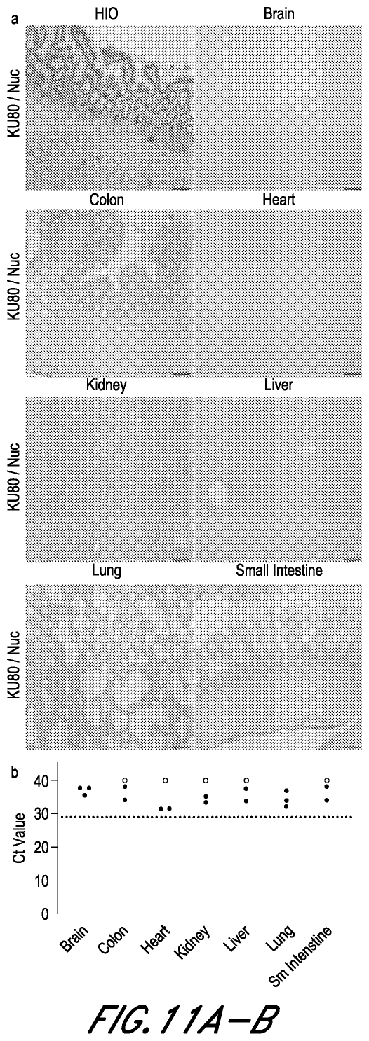

The biodistribution and potential expansion of the HI GFP cells within rats receiving fragmented HIOs as a cell therapy was investigated. Samples were collected from various off target organs from ten week postoperative rats. Portions of the brain, colon, heart, kidney, liver, lung, and small intestines were collected for histology and PCR. No gross abnormalities or tumor formation was observed in any of the harvested organs. When immunostaining for a human marker, KU80, across serial sections through 2 mm of tissue thickness, KU80+ cells were only observed in the transplanted HIO positive control (FIG. 11A-D). To further investigate human cell presence, the gold standard of Alu-based real-time PCR was performed to discern human from rodent cells. To determine the threshold for significance, a titration was performed on a known human gDNA sample and related back to cellular equivalence (Table 1). Then, experimental samples were run, including the H1 GFP cell line and the various off target organs. No Ct data points were found to be below the Ct value threshold for human cell presence (FIG. 11B) suggesting little to no human cell biodistribution within the experimental set up.

### Example 4. Methodology

Human Tissue: Human tissue collection was performed with the prior approval of an Institutional Review Board. Surgical samples of pathologically normal adult human small bowel and colon were obtained from patients undergoing bariatric or revision/resection procedures between the ages of 14 and 25 years old. Informed consent or assent was obtained from all patients and/or parent/legal guardians as appropriate. Additional de-identified samples of pathologically normal colon were obtained through the Discover Together Biobank of Cincinnati Children's Hospital Medical Center. All human tissue was utilized in accordance with institutional ethics guidelines.

Rats: All animal procedures and experiments were performed with the prior approval of an Institutional Animal Care and Use Committee. Both males and females were utilized for experiments. Adult immunodeficient rats with Rag1 and II2rg gene deletions (RRG) with ages between three and six months of age were used for mucosectomy experiments (founders from Transgenesis Rat ImmunoPhenomic Platform, Nantes, France, in-house breeding). Rats were housed in a barrier animal vivarium and handled humanely in accordance with the NIH Guide for the Care and Use of Laboratory Animals. RRG rats were primarily fed standard autoclaved chow and provided water bottles supplemented with fluconazole (0.1 mg/mL, NorthStar Rx, LLC). Both food and water were provided ad libitum before and after surgeries. A single dose of carprofen (5 mg/kg) was administered for pain management at the end of the mucosectomy procedure. Rats were monitored daily for three days postoperatively for signs of pain and distress and additional analgesics were administered as needed.

HI-GFP Cell Line Generation: CRISPR/Cas9 was used for introduction of the green fluorescent protein (GFP) sequence to the AAVS1 safe-harbor site in commercially available H1 human embryonic stem cells (hESCs, WiCell Research Institute, Inc.) using modified previously published reagents. Briefly, single-stranded donor oligonucleotides (ssODNs) encoding the validated guide RNA sequence (5′-GGGGCCACTAGGGACAGGAT-3′; SEQ ID NO: 1) for targeting the AAVS1 locus were annealed and subcloned into PX458M-HF, a modified version of pSpCas9 (BB)-2A-GFP (PX458; Addgene #48138) generated by the Cincinnati Children's Medical Center Transgenic Core, which carries an optimized single guide RNA. Transfection was performed using TransIT-LT1 transfection reagent according to the manufacturer's recommendations in mTeSR1® media containing 10 μM Y-276323 on hESC-qualified Matrigel®-coated plates (Corning®). Four hours post-transfection, media was removed and replaced with mouse embryonic fibroblast (MEF) conditioned hESC media (DMEM/F12, 20% knockout serum replacement (KOSR), 0.1 mM nonessential amino acids (NEAA), 2 mM L-glutamine, 0.1 mM β-mercaptoethanol and 4 ng/mL bFGF) containing 10 μM Y-27632 with daily media changes with MEF-conditioned hESC media. At 2 days post-transfection, a single-cell suspension of cells was generated with Accutase® (StemCell Technologies®) and replated at an approximate density of 10,000 cells/cm² for Geneticin® (G418) selection. Beginning three days post-transfection, G418 selection (100 μg/mL) was performed for eight days, after which daily feeds were performed using mTeSR1. After two weeks, the remaining G418-resistant colonies were harvested using Accutase and plated at cloning density in mTeSR® with CloneR® supplement (StemCell Technologies). Recovered clones were manually excised, expanded in mTeSRIR media, and subjected to genotyping. Correctly targeted insertion of the 2A-NeoR-CAG-GFP cassette in the AAVS1 locus was performed by PCR, Sanger sequencing, copy-number analysis and GFP expression. This cell line was used in all experiments. Monthly mycoplasma testing was performed using the MycoAlert Plus Detection Kit and Control Set (Lonza #LT07-705 & LT07-518) on all cell cultures and results were consistently negative.

Generation of Human Intestinal Organoids: Human intestinal organoids (HIOs) were generated and maintained as previously described (Watson et al. Nature Medicine (2014) 20:1310-1314; Spence et al. Nature (2011) 470:105-109; McCracken et al. Nature Protocols (2011) 6:1920-1928). Briefly, H1-GFP cells were grown in feeder-free conditions in Matrigel® (BD Biosciences®)-coated six-well Nunclon® surface plates (Nunc®) and maintained in mTeSR1® media (StemCell Technologies®). For definitive endoderm (DE) induction, cells were passaged as single cell suspensions generated with Accutase® (StemCell Technologies®) and plated in 24-well Nunc® plates at a density of approximately 100,000 cells/well. Cells grew in mTeSR1® media for two days before treatment with 100 ng/ml of Activin A for three days. DE was then treated with hindgut induction medium (RPMI 1640, 100×NEAA, 2% dialyzed fetal calf serum (dFCS) for four days with 100 ng/ml FGF4 (R&D Systems®) and 3 μM CHIRON 99021 (CHIR99021; Tocris®) to induce mid-hindgut spheroids. Spheroids were then plated in Growth Factor Reduced (GFR) Matrigel® and maintained in intestinal growth medium (Advanced DMEM/F12, N2 supplement, B27 supplement, 15 mM HEPES, 2 mM L-glutamine, penicillin-streptomycin) supplemented with 100 ng/mL EGF (R&D Systems) to generated HIOs. Media was changed twice weekly and HIOs were re-plated in fresh Matrigel® on day 14. HIOs were utilized for surgical transplantation between days 28 and 34.

Generation of Enteroids from Transplanted Human Intestinal Organoids: Crypts were isolated from transplanted HIOs as previously described (40). Briefly, segments of HIO tissue were pinned down in a SYLGARD 184 (Dow) coated petri dish, gently scraped to remove villi, washed with 2 mM chelation buffer before a 30 minute incubation in 2 mM chelation buffer. Then, to release the crypts the tissue was again gently scraped. The chelation buffer containing the crypts was removed from the petri dish, filtered through a 150 μm nylon mesh, and spun down at 50 g for 5 min at 4° C. to pellet the crypts for use in cell culture. Crypts were plated in Matrigel (Corning) and IntestiCult media (STEMCELL Technologies) was used to generate enteroids. Media was changed twice weekly and passages occurred every 7 to 10 days.

Mucosectomy Surgical Procedure: The mucosectomy procedure was optimized for the purposes here from a previously published study (Avansino et al. Surgery (2006) 140:423-434).

Loop Creation: One day prior to the procedure, chow diet was removed, and rats were placed on GelDiet 76A (ClearH₂O) to be continued seven days postoperatively before returning to chow. Rats were anesthetized with 2% inhaled isoflurane (Butler Schein), and their abdomen shaved and prepped in sterile fashion using swabs coated with isopropyl alcohol and povidone-iodine. A midline laparotomy of approximately 3 cm was made. A single dose of piperacillin and tazobactam (100 mg/kg) was administered within the abdominal cavity using an 18G blunt tip fill needle affixed to a 5 ml syringe. Then, the cecum was identified, and the intestine eviscerated, using saline warmed to 37° C. to maintain tissue moisture throughout the procedure. A suitable stretch of bowel (˜2-3 cm) was first identified for blind loop creation. To create the loop, the bowel and mesentery at the proximal end of what will become the loop was transfected using scissors and a Bovie pen, as necessary. Then, the distal end of the blind loop was partially transected using scissors. An anastomosis was performed using 7-0 silk suture (PERMA-HAND; Ethicon) between the open end of the fully transfected bowel and the partially transected distal end of the blind loop in a simple interrupted fashion. This established full continuity for the rat and maintains the blind loop's distal connection.

Injury Creation: A bulldog clamp was applied to the distal end of the blind loop, blocking flow into the continuous bowel. First, the chemical damage was induced. Using a 20 mL syringe equipped with a cannula, the loop was flushed with saline warmed to 37° C. for 2 min. Then, the loop was flushed with 1 mM dithiothreitol (DTT) in saline warmed to 37° C. for 2 min using a 20 mL syringe equipped with a cannula. The loop was subsequently flushed with warm saline again before flushing with 5 mM isotonic ethylenediamine tetra-acetic acid (EDTA) buffer warmed to 37° C. for 10 min using a 20 ml syringe equipped with a cannula. Approximately 50 mL of EDTA solution was used for flushing over the 10 min period. This series of flushes was repeated and then followed by a final flush was saline warmed to 37° C. To induce the mechanical damage, a dental go-between style brush flosser of appropriate diameter was inserted and removed three times, slightly twisting during entry and exit. After injury creation, the bulldog clamp was removed, and the distal end of the blind loop was tied off using 4-0 absorbable Chromic Gut suture (ETHILON; Ethicon).

Reseeding the Loop: Loops were reseeded with dissociated HIOs or media void of cellular content. To fragment structures, the HIOs were collected in their media and pooled. Then, the HIOs were drawn into a syringe affixed with a 18G blunt tip fill needle, the 18G needle was exchanged for a 20G needle, and the contents of the syringe were evacuated into a well of a 24 well plate. This process was repeated using sequentially smaller needles ending at 25G (FIG. 10A). Then, the HIO fragments were drawn into a 1 mL syringe equipped with a cannula and deposited within the prepared intestinal loop. The same process may also be done with enteroids (FIG. 10B). FIG. 10C shows a brightfield image of cells dissociated from HIOs or enteroids. The approximate number of cells that make up the original HIO or enteroid structure used for dissociation is shown in FIG. 10D. Approximately 100,000 cells per 4 mm of bowel length was used for reseeding with dissociated HIO. After reseeding, the proximal end of the loop was closed using 5-0 silk suture (PERMA-HAND; Ethicon) as the cannula was removed. The bowel was then carefully replaced within the abdominal cavity. To close the incision, the muscle was sutured in a running fashion using 4-0 coated absorbable suture (VICRYL RAPIDE; Ethicon). Then, the skin was closed in a buried interrupted fashion again using 4-0 coated absorbable suture (VICRYL RAPIDE; Ethicon). A single dose of carprofen (5 mg/kg) was administered for pain management at the end of the procedure. Tissues were harvested seven days postoperatively as an early time point during the healing process, and ten weeks postoperatively as a long term, recovered time point.

In vitro HIO Cell Quantification: Nuclei were isolated from HIOs using the Minute Detergent Free Nuclei Isolation Kit (Invent Biotechnologies, Inc.) following manufacturer's guidelines. To have sufficient cell quantities, structures were pooled for isolation. Four day 28 (d28) HIOs were pooled for single nuclear isolations. Immediately following completion, nuclei were automatically quantified using a TC20 (Bio-Rad Laboratories, Inc.). The total count was then divided by four to determine the number of cells per HIO.

Scanning Electron Microscopy: Segments of healthy rat jejunum and freshly damaged rat jejunum were fixed overnight in 3% glutaraldehyde in 0.175 M sodium cacodylate buffer pH 7.4. Samples were then buffer rinsed and post-fixed in 1% osmium tetroxide in 0.175 M cacodylate buffer for 1 hour at 4° C. After another buffer rinse, samples were put through a graded ethanol series (25%, 50%, 75%, 95%, 3×100%) for dehydration. Specimens were then critical point dried in an EM CPD300 (Leica®), stub mounted and sputter coated 10 nm thick with 60/40 gold/palladium using an EM ACE600 (Leica®). A SU8010 transmission electronic microscope (Hitachi®) was used to image samples.

Tissue Processing and Immunostaining: Samples were harvested and fixed overnight in 4% paraformaldehyde (PFA), processed and embedded in paraffin blocks. Sections were deparaffinized and either stained immediately with hematoxylin and eosin or subject to antigen retrieval, and antibody stained. Antibody incubations took place at 4° C. overnight in 1% bovine serum albumin in phosphate buffered saline (PBS). Antibodies and their respective dilutions are listed in Table 3. The Vectastain ABC system was used for amplification and the diaminobenzidine substrate kit was used for signal detection (Vector Laboratories®). Lillie-Mayer's Hematoxylin (Agilent Technologies®) was used as a counterstain. For biodistribution, serial sections were made and every tenth slide was stained over 2 mm of tissue thickness.

TABLE 1
Control Human DNA and determination of Ct
Value cutoff for human cellular presence.

| Hu DNA (ng) | Ct Value | Cell equivalent |
|---|---|---|
| 200 | 15.93 | 30303 |
| 20 | 17.35 | 3030 |
| 2 | 20.25 | 303 |
| 0.2 | 23.70 | 30 |
| 0.02 | 27.70 | 3 |
| 0.002 | 31.80 | 0.300 |
| 0.0002 | 35.10 | 0.03 |
| 0.00002 | 37.00 | 0.003 |
| Neg Control | 39.00 | 0 |

Titration of human DNA content in relation to Ct values and cell presence equivalency used for tumorigenicity.

TABLE 2
Human DNA Ct Values in rat tissues post mucosectomy.

| H1 GFP | | | Ct Value | | |
|---|---|---|---|---|---|
| DNA(ng) | Ct Value | Organ | Rat 1 | Rat 2 | Rat 3 |
| 200 | 17.4 | 200 ng DNA | | | |
| 20 | 20.1 | Brain | 37.7 | 37.7 | 35.5 |
| 2 | 23.7 | Colon | 34.1 | 38.2 | undetermined |
| 0.2 | 27.6 | Heart | 31.6 | 31.5 | undetermined |
| 0.02 | 31.3 | Kidney | 33.4 | 35.2 | undetermined |
| 0.002 | 35.1 | Liver | 33.8 | 37.6 | undetermined |
| 0.0002 | 37.7 | Lung | 36.9 | 32.1 | 33.9 |
| 0.00002 | 38.3 | Sm Intestine | 34.1 | 38.2 | undetermined |
| Neg Control | 38.0 | | | | |

Titration of human DNA content in the H1 GFP cell line used to generate HIOs and their associated Ct values. Quantification of Human DNA content by PCR throughout the major organs of rats having undergone mucosectomy procedures with fragmented HIOs used as a reseeding material. Undetermined Ct values are above 40 as per the sensitivity of the thermocycler. No values were found to be below the determined Ct threshold of 29, thus human cellular presence was not found within the samples.

TABLE 3
List of primary antibodies used for immunostaining

| | | | Catalogue |
|---|---|---|---|
| Antigen | Host | Supplier | No. |
| ACTA2; Actin Alpha 2, Smooth | mouse | Abcam | ab7817 |
| Muscle | | | |
| CDH1; Cadherin 1 | mouse | BD | 610182 |
| | | Biosciences | |
| CDH1; Cadherin 1 | goat | R&D | AF648 |
| DEFA5; Defensin Alpha 5 | mouse | Abcam | ab90802 |
| F3; Coagulation Factor III, | rabbit | Atlas | HPA049292 |
| Tissue Factor | | Antibodies | |
| GATA4; GATA Binding Protein | mouse | Santa Cruz | sc-25310 |
| 4 | | | |
| GFP; Green fluorescent protein | chicken | Abcam | ab13970 |
| KU80; Ku Autoantigen, 80 kDa | rabbit | Cell Signaling | 2180 |
| MKI67; Marker of Proliferation | rabbit | Thermo | RM-9106-S0 |
| Ki-67 | | | |
| MS4A12; Membrane Spanning | rabbit | Atlas | HPA057657 |
| 4-Domains A12 | | Antibodies | |
| MUC5B; Mucin 5B, Oligomeric | rabbit | Atlas | HPA008246 |
| Mucus/Gel-Forming | | Antibodies | |
| OLFM4; Olfactomedin 4 | rabbit | Cell Signaling | 14369 |
| SATB2; SATB Homeobox 2 | rabbit | Millipore | 384R-14 |
| | | Sigma | |
| SI; Sucrase-Isomaltase | rabbit | Atlas | HPA011897 |
| | | Antibodies | |
| TUBB3; Tubulin Beta 3 Class | chicken | Abcam | ab41489 |
| III | | | |

TABLE 4
List of secondary antibodies used for immunostaining

| Antigen | Host | Supplier | Catalogue No. |
|---|---|---|---|
| α-chicken biotin | goat | Vector Labs | BA-9010 |
| α-goat biotin | horse | Vector Labs | BA-9500 |
| α-mouse biotin | horse | Vector Labs | BA-2000 |
| α-rabbit biotin | goat | Vector Labs | BA-1000 |
| α-goat Alexa Fluor 647 | donkey | Invitrogen | A21447 |
| α-mouse Alexa Fluor 568 | donkey | Invitrogen | A10037 |
| α-rabbit Alexa Fluor 568 | donkey | Invitrogen | A10042 |
| α-rabbit Alexa Fluor 647 | donkey | Invitrogen | A31573 |

Image Acquisition: Surgical imagery was acquired using an M80 microscope outfitted with a MC 170HD camera (Leica Microsystems®). Gross images of harvested structures were acquired using a V40 ThinQ (LG Electronics®). Harvests were performed using a M165 FC microscope outfitted with a DCF7000 T camera (Leica Microsystems®). Slides were imaged using an Eclipse Ti microscope (Nikon Corporation®) and subsequent analysis performed using Nikon Element Imaging Software (Nikon Corporation®).

Ex vivo Epithelial Characteristics and Permeability: The epitheliums of freshly harvested healthy jejunum and reseeded loops were carefully dissected through a technique similar to seromuscular stripping as previously reported (Clarke, Am. J. Physiol. Gastrointest. Liver Physiol. (2009) 296: G1151-1166; Giles et al. Nat. Med. (2017) 23:829-838; Poling et al. Nat. Biomed. Eng. (2018) 2:429-442). Samples were opened and dissection was done in ice cold Kreb's buffer (117 mM NaCl; 4.7 mM KCl; 1.2 mM MgCl₂; 1.2 mM NaH₂PO₄; 25 mM NaHCO₃; 2.5 mM CaCl₂); 11 mM glucose). Full thickness tissue segments were then pinned in a dish containing cured Sylgard (Electron Microscopy Sciences). Reseeded loops were verified to be GFP positive before further dissection and use in the Ussing chamber assay. The seromusculature was then micro-dissected from the epithelium using Dumont #5 and #7 forcepts along with Vannas scissors (Fine Science Tools, Inc.). Gross tissue integrity was assessed using the stereoscope's bottom lighting for uniformity in appearance and any damaged areas were removed. Some remnant subepithelial mucosa remained after dissection. The central portion of the epithelium, with the least amount of handling, was then positioned for mounting between the hemi-chambers of an Ussing apparatus (Physiologic Instruments). 0.031 cm² of tissue was exposed to 5 mL of oxygenated Krebs buffer at 37° C. throughout the assay. The transepithelial potential difference was detected with two paired electrodes affixed to a salt bridge containing 3.75% agar in 3 M KCl. The electrodes were connected to a VVC MC8 voltage clamp amplifier (Physiologic Instruments). Electrode potential difference and fluid resistance values were offset to zero immediately before sliders were mounted between the chambers. A 30 min period was allowed for the establishment of equilibrium. Then, tissues were voltage-clamped at 0 mV while continuously measuring the short circuit current (I_{sc}) and chemical stimuli applied (10 μM forskolin, 100 μM IBMX, and 100 μM bumetanide). For FITC-dextran permeability, 2.2 mg/mL FITC-dextran was added into the apical side, and a sample was taken from the basolateral side every 30 minutes for 3 hours, replacing the same amount of fresh modified Kreb's buffer in the basolateral side to maintain pressure across the sample. Once all aqueous samples were collected, they were quantified with a plate-reader (Synergy 2, BioTek).

Human Specific Alu PCR Primers and Probe: Detection of human DNA was done using previously described primers and probe. Briefly, Alu PCR was performed on gDNA extracted from various organs from reseeded mucosectomy rats and the H1 GFP cell line. The forward primer was designed to anneal upstream of the human specific Alu sequence (5′-TGGTGG CTCTCT CCT GTA AT-3′; SEQ ID NO: 2) and the reverse primer was designed to primarily anneal within the human-specific Alu sequence (5′-GAT CTC GGC TCA CTG CAA C-3′; SEQ ID NO: 3), resulting in a 96 base pair amplicon. The probe was designed to bind between the two primers (5′-TGA GGC AGG AGA ATC GCT TGA ACC-3′; SEQ ID NO: 4) quencher-MGB-6FAM upstream of the hAlu-specific sequence. The primers and probes were custom ordered from Integrated DNA Technologies.

Alu PCR: Quantitative real time PCR was performed using TaqMan Universal PCR Master Mix (Applied Biosystems) on 200 ng of target template gDNA. Each sample was sequenced in triplicate using a OneStep thermocycler (Applied Biosystems). Standard curves were generated by adding 10-fold serial dilutions (200 ng-0 ng) of hDNA (Millipore Sigma) and H1 GFP cells on each PCR plate. QunatoStudio software (Applied Biosystems) was used to calculate crossing threshold (Ct) values for presence of human cells based on the standard values.

Data Representation, Statistics and Reproducibility: For bar growths, data is represented as a mean±standard deviation, with all individual data points represented. For violin plots, a dashed line indicates the mean and interquartile ranges, with all individual data points represented. For statistics comparing two groups of paired data, Wilcoxon Signed-Ranks tests were performed. For statistics comparing unpaired data, a Student's t-test was performed. The statistical significance cutoff was p<0.05 and confidence interval of 95%.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” is typically interpreted as “including but not limited to,” the term “having” is typically interpreted as “having at least,” the term “includes” is typically interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases is typically construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” is typically interpreted to mean “at least one” or “one or more”); the same holds true for the use of definite articles used to introduce claim recitations. In addition, even if a specific number of an introduced claim recitation is explicitly recited, those skilled in the art will recognize that such recitation is typically interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, is typically understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

H Vallicelli, C. et al. Small bowel emergency surgery: literature's review. World journal of emergency surgery: WJES 6, 1 (2011).
Watson, C. L. et al. An in vivo model of human small intestine using pluripotent stem cells. Nature medicine 20, 1310-1314 (2014).
Singh, A. et al. Evaluation of transplantation sites for human intestinal organoids. PloS one 15, e0237885 (2020).
Menoret, S. et al. Generation of Immunodeficient Rats With Rag1 and II2rg Gene Deletions and Human Tissue Grafting Models. Transplantation 102, 1271-1278 (2018).
Tait, I. S., Evans, G. S., Flint, N. & Campbell, F. C. Colonic mucosal replacement by syngeneic small intestinal stem cell transplantation. American journal of surgery 167, 67-72 (1994).
Allard, J. et al. Immunohistochemical toolkit for tracking and quantifying xenotransplanted human stem cells. Regenerative medicine 9, 437-452 (2014).
Qin, X. Why is damage limited to the mucosa in ulcerative colitis but transmural in Crohn's disease? World J Gastrointest Pathophysiol 4, 63-64 (2013).
Avansino, J. R., Chen, D. C., Hoagland, V. D., Woolman, J. D. & Stelzner, M. Orthotopic transplantation of intestinal mucosal organoids in rodents. Surgery 140, 423-434 (2006).
Vannucchi, M. G. The Telocytes: Ten Years after Their Introduction in the Scientific Literature. An Update on Their Morphology, Distribution, and Potential Roles in the Gut. Int J Mol Sci 21 (2020).
Kinchen, J. et al. Structural Remodeling of the Human Colonic Mesenchyme in Inflammatory Bowel Disease. Cell 175, 372-386 e317 (2018).
Thompson, C. A. et al. GATA4 Is Sufficient to Establish Jejunal Versus Ileal Identity in the Small Intestine. Cell Mol Gastroenterol Hepatol 3, 422-446 (2017).
Bykov, V. L. [Paneth cells: history of discovery, structural and functional characteristics and the role in the maintenance of homeostasis in the small intestine]. Morfologiia 145, 67-80 (2014).
Galand, G. Brush border membrane sucrase-isomaltase, maltase-glucoamylase and trehalase in mammals. Comparative development, effects of glucocorticoids, molecular mechanisms, and phylogenetic implications. Comp Biochem Physiol B 94, 1-11 (1989).
Munera, J. O. et al. Differentiation of Human Pluripotent Stem Cells into Colonic Organoids via Transient Activation of BMP Signaling. Cell Stem Cell 21, 51-64 e56 (2017).
Koslowski, M., Sahin, U., Dhaene, K., Huber, C. & Tureci, O. MS4A12 is a colon-selective store-operated calcium channel promoting malignant cell processes. Cancer research 68, 3458-3466 (2008).
van Klinken, B. J. et al. MUC5B is the prominent mucin in human gallbladder and is also expressed in a subset of colonic goblet cells. Am J Physiol 274, G871-878 (1998).
Clarke, L. L. A guide to Ussing chamber studies of mouse intestine. American journal of physiology. Gastrointestinal and liver physiology 296, G1151-1166 (2009).
Ma, T. Y., Hollander, D., Bhalla, D., Nguyen, H. & Krugliak, P. IEC-18, a nontransformed small intestinal cell line for studying epithelial permeability. J Lab Clin Med 120, 329-341 (1992).
Srinivasan, B. et al. TEER measurement techniques for in vitro barrier model systems. J Lab Autom 20, 107-126 (2015).
Finkbeiner, S. R. et al. Transcriptome-wide Analysis Reveals Hallmarks of Human Intestine Development and Maturation In Vitro and In Vivo. Stem cell reports (2015).
Yui, S. et al. Functional engraftment of colon epithelium expanded in vitro from a single adult Lgr5 (+) stem cell. Nature medicine 18, 618-623 (2012).
Nakamura, T. & Watanabe, M. Intestinal stem cell transplantation. J Gastroenterol 52, 151-157 (2017).
Fukuda, M. et al. Small intestinal stem cell identity is maintained with functional Paneth cells in heterotopically grafted epithelium onto the colon. Genes & development 28, 1752-1757 (2014).
Fordham, R. P. et al. Transplantation of expanded fetal intestinal progenitors contributes to colon regeneration after injury. Cell Stem Cell 13, 734-744 (2013).
Sugimoto, S. et al. Reconstruction of the Human Colon Epithelium In Vivo. Cell Stem Cell 22, 171-176 e175 (2018).
Khalil, H. A. et al. Intestinal epithelial replacement by transplantation of cultured murine and human cells into the small intestine. PloS one 14, e0216326 (2019).
Agopian, V. G., Chen, D. C., Avansino, J. R. & Stelzner, M. Intestinal stem cell organoid transplantation generates neomucosa in dogs. J Gastrointest Surg 13, 971-982 (2009).
Sugimoto, S. et al. An organoid-based organ-repurposing approach to treat short bowel syndrome. Nature (2021).
Tsai, Y. H. et al. In vitro patterning of pluripotent stem cell-derived intestine recapitulates in vivo human development. Development 144, 1045-1055 (2017).
Oceguera-Yanez, F. et al. Engineering the AAVS1 locus for consistent and scalable transgene expression in human iPSCs and their differentiated derivatives. Methods 101, 43-55 (2016).
Ran, F. A. et al. Genome engineering using the CRISPR-Cas9 system. Nature protocols 8, 2281-2308 (2013).
Chen, B. et al. Dynamic imaging of genomic loci in living human cells by an optimized CRISPR/Cas system. Cell 155, 1479-1491 (2013).
Spence, J. R. et al. Directed differentiation of human pluripotent stem cells into intestinal tissue in vitro. Nature 470, 105-109 (2011).
McCracken, K. W., Howell, J. C., Wells, J. M. & Spence, J. R. Generating human intestinal tissue from pluripotent stem cells in vitro. Nature protocols 6, 1920-1928 (2011).
Giles, D. A. et al. Thermoneutral housing exacerbates nonalcoholic fatty liver disease in mice and allows for sex-independent disease modeling. Nature medicine 23, 829-838 (2017).
Poling, H. M. et al. Mechanically induced development and maturation of human intestinal organoids in vivo. Nat Biomed Eng 2, 429-442 (2018).
Lanas, A., Chan F. K. L., Peptic ulcer disease. Lancet 390, 613-624 (2017).
Kuna, G. L. et al., Peptic Ulcer Disease: A Brief Review of Conventional Therapy and Herbal Treatment Options. J Clin Med 8, (2019).
Wong, L. et al., High incidence of mortality and recurrent bleeding in patients with Helicobacter pylori-negative idiopathic bleeding ulcers. Gastroenterology 137, 525-531 (2009).
Singh, A., Poling, H. M., Spence J. R., Wells, J. M., Helmrath, M. A., Gastrointestinal organoids: a next-generation tool for modeling human development. Am J Physiol Gastrointest Liver Physiol 319, G375-G381 (2020).
Sugimoto, S., et al., An organoid-based organ-repurposing approach to treat short bowel syndrome. Nature, (2021).
Mahe, M. M., Sundaram, N., Watson, C. L., Shroyer, N. F., Helmrath, M. A., Establishment of human epithelial enteroids and colonoids from whole tissue and biopsy. J Vis Exp, (2015).

1. A method of treating intestinal damage of a subject in need thereof, comprising administering a dissociated cell population that is dissociated from intestinal and/or colonic organoids to the luminal wall of the intestine of the subject, wherein the cell population dissociated from the intestinal and/or colonic organoids comprise epithelial cell types and mesenchymal cell types, and wherein the intestine of the subject comprises the small intestine and/or the colon.

2. The method of claim 1, wherein administering the dissociated cell population to the luminal wall of the intestine of the subject comprises administering the cell population to a location of the lumen of the intestine affected by the intestinal damage, optionally wherein the location is directly adjacent to or near the intestine affected by the intestinal damage, optionally to the surface of the luminal wall.

3. The method of claim 1, wherein the dissociated cell population is administered to the luminal wall of the intestine of the subject as a cell suspension; and/or wherein administering the dissociated cell population to the luminal wall of the intestine of the subject comprises administering the cell population by oroenteric catheter, nasoenteric catheter, or enema.

4. (canceled)

5. The method of claim 1, wherein the intestinal and/or colonic organoids have been derived from precursor cells selected from embryonic stem cells, induced pluripotent stem cells, and definitive endoderm cells.

6. The method of claim 1, wherein the intestinal and/or colonic organoids are allogeneic to the subject.

7. The method of claim 1, wherein the intestinal and/or colonic organoids have been derived from cells isolated from the subject, and the intestinal and/or colonic organoids are autologous to the subject; and/or wherein the intestinal and/or colonic organoids have been derived from induced pluripotent stem cells derived from the cells isolated from the subject, optionally wherein the cells isolated from the subject comprise dermal fibroblasts or peripheral blood mononuclear cells (PBMCs) from the subject.

8. (canceled)

9. (canceled)

10. The method of claim 1, wherein the dissociated cell population is prepared by enzymatic dissociation and/or mechanical dissociation of the intestinal and/or colonic organoids.

11. (canceled)

12. (canceled)

13. The method of claim 1, wherein the percentage of cells in the dissociated cell population that are mesenchymal cell types is or is about 85%, 86%, 87%, 88%, 89%, 90%, 91%, 92%, 93%, 94%, or 95%, or any percentage within a range defined by any two of the aforementioned percentages; and/or wherein the percentage of cells in the dissociated cell population that are epithelial cell types is or is about 5%, 6%, 7%, 8%, 9%, 10%, 11%, 12%, 13%, 14%, or 15%, or any percentage within a range defined by any two of the aforementioned percentages.

14. (canceled)

15. The method of claim 1, wherein the dissociated cell population is administered at a concentration that is or is about 50, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 cells per mm² of affected intestine surface area, or any amount of cells per mm² within a range defined by any two of the aforementioned values.

16. (canceled)

17. (canceled)

18. The method of claim 1, wherein cells of the dissociated cell population integrate into the mucosa and muscularis of the intestine of the subject; and/or wherein cells of the dissociated cell population integrated into the intestine of the subject maintain their intestinal and/or colonic regionality; and/or a subpopulation thereof, of the dissociated cell population integrated into the intestine of the subject differentiate into smooth muscle actin (SMA)-positive smooth muscle cell types; and/or wherein the dissociated cell population comprises Marker of Proliferation KI67+ (MKI67+) proliferative cells that integrate into the intestine of the subject and promote healing of the intestinal damage; and/or wherein the dissociated cell population promotes formation of an intact intestinal barrier after administration.

19. (canceled)

20. (canceled)

21. (canceled)

22. (canceled)

23. The method of claim 1, wherein the dissociated cell population integrates into at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% of the surface area of the luminal wall of the intestine of the subject affected by the intestinal damage, or any percentage of surface area within a range defined by any two of the aforementioned percentages.

24. The method of claim 1, wherein the intestinal damage comprises intestinal ulceration; and/or wherein the intestinal damage is chemical and/or mechanical and/or or is associated with a gastrointestinal malady.

25. (canceled)

26. (canceled)

27. The method of claim 24, wherein the gastrointestinal malady is selected from Crohn's disease, ulcerative colitis, enteropathies associated with non-steroidal anti-inflammatory drugs (NSAIDs) or other medications, radiation-induced enteropathies, and enteropathies associated with pathogenic infections such as tuberculosis.

28. The method of claim 1, wherein the intestinal and/or colonic organoids are mammalian; and/or wherein the subject is mammalian.

29. The method of claim 1, wherein the intestinal and/or colonic organoids are human; and/or wherein the subject is human.

30. (canceled)

31. (canceled)

32. The method of claim 1, further comprising dissociating the intestinal and/or colonic organoids to produce the dissociated cell population.

33. The method of claim 1, wherein the dissociated cell population is in the form of 30%, 35%, 40%, 45%, 50%, 55%, 60%, 65%, 70%, 75%, 80%, 85%, 90%, 95%, 96%, 97%, 98%, 99%, or 100% multi-cellular fragments, or in the form of at least 30%, 35%, 40%, 45%, 50%, 55%, 60%, 65%, 70%, 75%, 80%, 85%, 90%, 95%, 96%, 97%, 98%, 99%, or 100% multi-cellular fragments.

34. The method of claim 1, further comprising producing the intestinal and/or colonic organoids in vitro, optionally from pluripotent stem cells.

35. A dissociated cell population dissociated from intestinal and/or colonic organoids for use in the method of claim 1.

36. An in vitro cell suspension comprising a dissociated cell population comprising epithelial cell types and mesenchymal cell types.

37. The cell suspension of claim 36, wherein the mesenchymal cell types express vimentin (VIM) and/or elastin microfibril interfacer 1 (EMILIN1); and the epithelial cell types express E-cadherin (CDH1) and/or caudal type homeobox 2 (CDX2).

38. The cell suspension of claim 36, wherein the dissociated cell population is dissociated from intestinal and/or colonic organoids, wherein the intestinal and/or colonic organoids comprise epithelial cell types and mesenchymal cell types; optionally wherein the intestinal and/or colonic organoids have been derived from precursor cells selected from embryonic stem cells, induced pluripotent stem cells, and definitive endoderm cells.

39. (canceled)

40. The cell suspension of claim 36, wherein the cell suspension or the intestinal and/or colonic organoids are allogeneic to a subject.

41. The cell suspension of claim 36, wherein the cell suspension or the intestinal and/or colonic organoids have been derived from cells from a subject, and the intestinal and/or colonic organoids are autologous to the subject; optionally wherein the cell suspension or the intestinal and/or colonic organoids have been derived from induced pluripotent stem cells derived from the cells isolated from the subject.

42. (canceled)

43. The cell suspension of claim 36, wherein the dissociated cell population is prepared by enzymatic dissociation and/or mechanical dissociation of the intestinal and/or colonic organoids.

44. (canceled)

45. (canceled)

46. The cell suspension of claim 35, wherein the dissociated cell population comprises MK167+ proliferative cells.

47. The cell suspension of claim 36, wherein the percentage of cells in the dissociated cell population that are mesenchymal cell types is at least 85%, 86%, 87%, 88%, 89%, 90%, 91%, 92%, 93%, 94%, or 95%, or a percentage within a range defined by any two of the aforementioned percentages; and/or the percentage of cells in the dissociated cell population that are epithelial cell types is or is about 5%, 6%, 7%, 8%, 9%, 10%, 11%, 12%, 13%, 14%, or 15%, or any percentage within a range defined by any two of the aforementioned percentages.

48. (canceled)

49. The cell suspension of claim 35, wherein the concentration of the dissociated cell population in the cell suspension is about 10⁵, 10⁶, 10⁷, 10⁸, 10⁹, 10¹⁰, or 10¹¹ cells/mL, or any concentration of cells within a range defined by any two of the aforementioned concentrations; and/or wherein the concentration of cells in the dissociated cell population that are mesenchymal cell types is about 10⁵, 10⁶, 10⁷, 10⁸, 10⁹, 10¹⁰, or 10¹¹ cells/mL, or any concentration of cells within a range defined by any two of the aforementioned concentrations; and/or wherein the dissociated cell population is in the form of, or of at least, 30%, 35%, 40%, 45%, 50%, 55%, 60%, 65%, 70%, 75%, 80%, 85%, 90%, 95%, 96%, 97%, 98%, 99%, or 100% multi-cellular fragments, or any percentage within a range defined by any two of the aforementioned percentages.

50. (canceled)

51. (canceled)

52. A pharmaceutical composition comprising an effective amount of the cell suspension of claim 36, and at least one pharmaceutically acceptable carrier, excipient, or diluent.

53. (canceled)


SEQUENCE LISTING (4 sequences)

SEQ ID NO: 1 - DNA, 20 bp, synthetic construct
  misc_feature 1..20: note = Human AAVS1 locus gRNA
  source 1..20: mol_type = other DNA
ggggccactagggacaggat

SEQ ID NO: 2 - DNA, 20 bp, synthetic construct
  source 1..20: mol_type = other DNA
  misc_feature 1..20: note = Human-specific Alu forward primer
tggtggctctctcctgtaat

SEQ ID NO: 3 - DNA, 19 bp, synthetic construct
  source 1..19: mol_type = other DNA
  misc_feature 1..19: note = Human-specific Alu reverse primer
gatctcggctcactgcaac

SEQ ID NO: 4 - DNA, 24 bp, synthetic construct
  source 1..24: mol_type = other DNA
  misc_feature 1..24: note = Human-specific Alu PCR probe
tgaggcaggagaatcgcttgaacc